(19) United States
(12) Patent Application Publication
(10) Pub. No.: US 2010/0247517 A1
(43) Pub. Date: Sep. 30, 2010

(54) USE OF MNK INHIBITORS FOR THE TREATMENT OF ALZHEIMER'S DISEASE

(75) Inventors: Matthias Austen, Goettingen (DE); Marcus Geese, Goettingen (DE); Martin Schneider, Goettingen (DE)

Correspondence Address: ROTHWELL, FIGG, ERNST & MANBECK, P.C., 1425 K STREET, N.W., SUITE 800, WASHINGTON, DC, 20005, US

(73) Assignee: Boehringer Ingelheim Internationational GmbH, Ingelheim am Rhein (DE)

(21) Appl. No.: 12/744,374

(22) PCT Filed: Nov. 21, 2008
(86) PCT No.: PCT/EP08/09880

§ 371 Date: May 24, 2010

(30) Foreign Application Priority Data
Nov. 22, 2007 (EP) 07022695.6

(51) Int. Cl. A61K 39/395; C07K 16/18; C07K 16/40; C07H 21/04; C07H 21/02; C07D 487/04; C07D 495/04; C12Q 1/48; A61K 31/7088; A61K 31/519; A61P 25/28

(52) U.S. Cl. 424/130.1; 530/387.1; 536/24.5; 544/262; 544/278; 435/15; 514/44 A; 514/262.1

(57) ABSTRACT

The present invention relates to the use of modulators of the kinase activity of Mnk1 and/or Mnk2 the diagnosis, alleviation, treatment and/or prevention of a tauopathy. Particularly, the present invention relates to the use of a modulator of Mnk1 and/or Mnk2 kinase for the diagnosis, alleviation, treatment and/or prevention of Alzheimer's disease. Preferably, the compounds are condensed pyrimidines.

The present invention relates to the use of modulators of the kinase activity of Mnk1 and/or Mnk2 for the diagnosis, alleviation, treatment and/or prevention of a tauopathy. Particularly, the present invention relates to the use of a modulator of Mnk1 and/or Mnk2 kinase for the diagnosis, alleviation, treatment and/or prevention of Alzheimer's disease.

Alzheimer's disease is a progressive neurodegenerative disease characterized by progressive cognitive decline, memory deterioration, neuropsychiatric disturbances and behavioural changes (Cummings, 2004). The incidence of Alzheimer's disease increases with age, such that 1% of 60 year olds and 30% of 85 year olds have the disease (Cummings, 2004). Combined with an increasing age of the population, the resulting progressive increase in Alzheimer cases represents a threatening burden for current healthcare systems. Currently marketed drugs for Alzheimer only show moderate symptomatic improvements at best; there are no true disease-modifying treatments on the market (Mount and Downton, 2006; Roberson and Mucke, 2006).

Characteristic hallmarks of Alzheimer disease pathology (and first described by Alzheimer 100 years ago) at the tissue level are extracellular plaques and intracellular neurofibrillary tangles (NFTs) as well as loss of neurons due to increased cell death (Goedert and Spillantini, 2006). Plaques have been shown to be aggregates consisting mostly of beta amyloid peptide (Aβ42), a protein fragment generated by processing of the APP transmembrane protein. NFTs are aggregates of hyperphosphorylated Tau, a microtubule binding protein (Goedert and Spillantini, 2006). In both cases, it is possible that cells are harmed not by large aggregates but rather by smaller, oligomeric protein complexes (SantaCruz et al., 2005; Gomez-Isla et al., 1997; Lesne et al., 2006; Jacobsen et al., 2006).

After many years of intensive focus on beta amyloid-containing extracellular plaques it is now appreciated that pathological changes in Tau posttranslational modification, expression and aggregation play a key role in several neurodegenerative diseases including Alzheimer's disease (Goedert and Spillantini, 2006; Kins and Beyreuther 2006). Phosphorylation of Tau at specific amino acids leads to dissociation from microtubules and thereby destabilizes axonal microtubule networks, causing a disruption of essential transport processes into distal parts of neurons (Kins and Beyreuther 2006; Biernat et al., 1993).

Highly instructive in the investigation of the role of Tau in neurodegeneration were socalled tauopathys—diseased in which changes in Tau lead to neurodegeneration. One important example for such a Tau-mediated neurodegenerative disease is frontotemporal dementia and parkinsonism linked to chromosome 17 (FTDP-17). More than 30 disease-causing missense mutations in Tau have been described in FTDP-17, most of which cluster in the repeat region of Tau, a region important for microtubule binding (Goedert and Spillantini, 2006). Most missense mutations in Tau lead to dissociation of Tau from microtubules, while only some also cause aggregation (Hasegawa et al., 1998, Goedert and Spillantini, 2006), indicating that impaired Tau-microtubule interaction can have a causative role in neurodegeneration (Kins and Beyreuther, 2006).

Ser262 of human Tau is an example for a phosphorylation site located in a central repeat where phosphorylation leads to dissociation of Tau from microtubules and altered microtubule dynamics in microtubule-Tau coincubation assays in vitro (Biernat et al., 1993; Drewes et al., 1995; Mandelkow et al., 1995). Phosphorylation of Ser262 is significantly increased in brain tissue from human Alzheimer patients and may be an early event in disease progression (Hasegawa et al., 1992; Augustinack et al., 2002). Indeed, in all neurodegenerative diseases, in which tau pathology has been observed, the tau is abnormally phosphorylated (Lee et al., 2001).

Further, phosphorylation of eIF4E is strongly increased in brain tissue of patients with Alzheimer's disease, and eIF4E brain phosphorylation levels positively correlate with disease severity (Li et al., 2004). Mnk1 and Mnk2 are the only relevant eIF4E kinases in vivo (Ueda et al., 2004), strongly suggesting that the activity of Mnk1, −2 or both is elevated in the brain of Alzheimer patients. Mnk activity is also increased by inflammatory factors which have also been implicated in Alzheimer's disease (Eikelenboom et al., 2006; Buxade et al., 2005). However, whether a rise in Mnk activity is just an inconsequential byproduct of neuronal derangements in Alzheimer's disease or whether it has a role in disease initiation and/or progression has not been investigated.

Therefore, the technical problem underlying the present invention was to provide for means and methods for the diagnosis, alleviation, treatment and/or prevention of neurodegenerative diseases in which there is a tauopathology. In particular, the technical problem of the invention was the provision of means and methods for the diagnosis, alleviation, treatment and/or prevention of a pathological condition caused by hyperphosphorylation of tau protein, in particular including an Alzheimer's disease.

The solution of the above technical problem is achieved by providing the embodiments characterised in claims.

The approach of the inventors of the present application was the identification and characterisation of protein kinases which can be linked to the increased phosphorylation of the tau protein, and in particular to the increased phosphorylation of amino acid residues within the microtubule binding region of tau. The phosphorylation of Ser262 and/or Ser356 within the microtubule binding region was of particular interest. In fact, the identification of protein kinases that phosphorylate tau in the pathological process of the tauopathies could provide potential therapeutic targets for the treatment of neurodegenerative diseases in which there is tauopathology. In particular, the identification of such a protein kinase could provide a potential therapeutic target for the treatment of Alzheimer's disease.

Protein kinases are important enzymes involved in the regulation of many cellular functions. The LK6-serine/threonine-kinase gene of Drosophila melanogaster was described as a short-lived kinase which can associate with microtubules (J. Cell Sci. 1997, 110(2): 209-219). Genetic analysis in the development of the compound eye of Drosophila suggested a role in the modulation of the RAS signal pathway (Genetics 2000 156(3): 1219-1230). The closest human homologues of Drosophila LK6-kinase are the MAP-kinase interacting kinase 2 (Mnk2, e.g. the variants Mnk2a and Mnk2b) and MAP-kinase interacting kinase 1 (Mnk1) and variants thereof. These kinases are mostly localised in the cytoplasm. Mnks are phosphorylated by the p42 MAP kinases Erk1 and Erk2 and the p38-MAP kinases. This phosphorylation is triggered in a response to growth factors, phorbol esters and oncogenes such as Ras and Mos, and by stress signalling molecules and cytokines. The phosphorylation of Mnk proteins stimulates their kinase activity towards eukaryotic initiation factor 4E (eIF4E) (EMBO J. 16: 1909-1920, 1997; Mol Cell Biol 19, 1871-1880, 1990; Mol Cell Biol 21, 743-754, 2001). Simultaneous disruption of both, the Mnk1 and Mnk2 gene in mice diminishes basal and stimulated eIF4E phosphorylation (Mol Cell Biol 24, 6539-6549, 2004). Phosphorylation of eIF4E results in a regulation of the protein translation (Mol Cell Biol 22: 5500-5511, 2001).

The co-owned patent application WO 03/037362 discloses the link between human Mnk genes, particularly human Mnk1 and Mnk2 genes, and diseases which are associated with the regulation of body weight or thermogenesis. It is postulated that human Mnk genes, particularly the Mnk2 and Mnk1 are involved in diseases such as e.g. metabolic diseases including obesity, eating disorders, cachexia, diabetes mellitus, hypertension, coronary heart disease, hypercholesterolemia, dyslipidemia, osteoarthritis, biliary stones, cancer of the genitals and sleep apnea, and in diseases connected with the ROS defense, such as e.g. diabetes mellitus and cancer. WO 03/03762 moreover discloses the use of nucleic acid sequences of the MAP kinase-interacting kinase (Mnk) gene family, in particular of Mnk1 and Mnk2, and amino acid sequences encoding these and the use of these sequences or of effectors of Mnk nucleic acids or polypeptides, particularly Mnk inhibitors and activators in the diagnosis, prophylaxis or therapy of diseases associated with the regulation of body weight or thermogenesis.

So far, it has not been described that Mnk kinases and in particular Mnk1 and/or Mnk2 are directly involved in the hyperphosphorylation of tau protein and thus may be associated with tau pathologies. In this application, the inventor showed that recombinant human tau protein is a substrate for recombinant human Mnk1 and/or Mnk2 in vitro. In particular, the inventors of the present invention found that Mnk1 and/or Mnk2 can phosphorylate Ser262 and Ser356 in human tau, both of which are located in the central repeat domains responsible for binding to microtubulis and for tau aggregation. It has therefore been surprisingly found that an increased activity of Mnk1 and/Mnk2 kinase can lead to increased phosphorylation of Ser262 and/or Ser356, which causes disruption of neuronal microtubule networks and consequent development of neurodegeneration.

Based on these findings, the inventors of the present invention could identify modulators of the Mnk1 and/or Mnk2 kinase, which may be useful in the diagnosis, alleviation, treatment and/or prevention of neurodegenerative diseases in which there is a tau pathology.

Accordingly, the object of the present invention is the use of a modulator of Mnk1 and/or Mnk2 kinase for the manufacture of an agent for the diagnosis, alleviation, treatment and/or prevention of a tauopathy. In a very preferred embodiment, the modulator of Mnk1 and/or Mnk2 kinase of the invention is for the diagnosis, alleviation, treatment and/or prevention of Alzheimer's disease.

Mnk-homologous proteins and nucleic acid molecules coding therefore, particularly human Mnk-homologous polypeptides and nucleic acids encoding such peptides are disclosed in the co-owned patent application WO 03/037362, which is herein incorporated by reference. In particular, this co-owned patent application discloses polypeptides and nucleic acids encoding a human Mnk2 protein and variants Mnk2a and Mnk2b as well as a human Mnk1 protein and variants Mnk1a and Mnk1b.

A modulator of Mnk1 and/or Mnk2 kinase or variants thereof according to the present invention may be any compound which alters, reduces or inhibits the activity of the kinase. In a very preferred embodiment of the invention, the modulator of the Mnk1 and/or Mnk2 kinase is a compound which reduces or inhibits the activity of the kinase.

Preferred modulators of Mnk1 and/or Mnk2 kinase can be selected from the group consisting of an antibody or antibody fragment against Mnk1 and/or Mnk2 kinase, an antisense molecule, a ribozyme or an RNAi molecule and/or a low molecular weight organic molecule.

Hence, the modulator of the Mnk1 and/or Mnk2 kinase of the present invention may be a binding molecule compound, which directly interacts with the kinase molecule as for example an antibody or an antibody fragment against the Mnk1 and/or Mnk2 kinase. The antibody of the invention can be a monoclonal or polyclonal antibody, preferably a monoclonal antibody. The antibody of the invention can be a whole antibody or an antigen-binding fragment, such as a Fab or F(ab′)₂ fragment, which contains specific binding sites for Mnk1 and/or Mnk2. The antibody may also be a recombinant antibody, i.e. a single-chain antibody or a fragment thereof, i.e. an scFv fragment.

The modulator of the Mnk1 and/or Mnk2 kinase can, on the other hand, act e.g. on the nucleic acid levels such as antisense nucleic acids, siRNA molecules and/or ribozymes.

Finally, according to a preferred embodiment of the present invention, the modulators of the activity of Mnk1 and/or Mnk2 kinase are selected from compounds which inhibit and/or reduce the kinase activity of Mnk1 and/or Mnk2. In a very preferred embodiment, the modulator of the present invention is an inhibitor of the kinase activity of Mnk1 and/or Mnk2.

Preferred modulators, in particular inhibitors of the kinase activity of Mnk1 and/or Mnk2 or variants thereof such as Mnk1a, Mnk1b, Mnk2a or Mnk2b are described in the co-owned international patent applications WO 2006/066937 filed on 22 Dec. 2005 and PCT/EP2006/005980 filed on 21 Jun. 2006, which are both herein incorporated as references.

Particularly preferred compounds for modulating (preferably inhibiting) the kinase activity of Mnk1 and/or Mnk2 or variants thereof are pyrazolopyrimidine compounds of the general Formula (I)

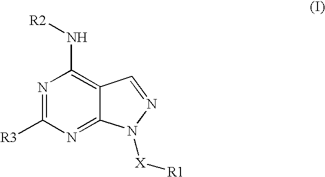

wherein R¹ is substituted aryl having 6 to 10 carbon atoms or optionally substituted heteroaryl having 5 to 10 ring atoms, wherein the substituents are one or more of R⁴, wherein R⁴ is independently halogen; CN; COOR⁵; OR⁵; C(O)N(R⁵R^{5a}); S(O)₂N(R^{S}R^{5a}); S(O)N(R⁵R^{5a}); S(O)₂R⁵; N(R⁵)S(O)₂N(R⁵R^{5a}); SR⁵; N(R⁵R^{5a}); OC(O)R⁵; N(R⁵)C(O)R^{5a}; N(R⁵)S(O)₂R^{5a}; N(R⁵)S(O)R^{5a}; N(R⁵)C(O)N(R^{5a}R^{5b}); N(R⁵)C(O)OR^{5a}; OC(O)N(R⁵R^{5a}); oxo (═O), where the ring is at least partially saturated; C(O)R⁵; T¹; or C₁₋₆ alkyl, wherein C₁₋₆ alkyl is optionally substituted with one or more R⁶;

R⁵, R^{5a} and R^{5b} are independently selected from the group consisting of H; T¹; and C₁₋₆ alkyl, wherein C₁₋₆ alkyl is optionally substituted with one or more R⁷;

R⁶, R⁷ are independently selected from the group consisting of halogen; CN; COOR⁸; OR⁸; C(O)R⁸; C(O)N(R⁸R^{8a}); S(O)₂N(R⁸R^{8a}); S(O)N(R⁸R^{8a}); S(O)₂R⁸; N(R⁸)S(O)₂N(R⁸¹R^{8b}); SR⁸; N(R⁸R^{8a}); OC(O)R⁸; N(R⁸)C(O)R^{8a}; N(R⁸)S(O)₂R^{8a}; N(R⁸)S(O)R^{8a}; N(R⁸)C(O)N(R^{8a}R^{8b}); N(R⁸)C(O)OR^{8a}; OC(O)N(R⁸R^{8a}); and T¹;

R⁸, R^{8a}, R^{8b} are independently selected from the group consisting of H; C₁₋₆ alkyl; and T¹;

wherein T¹ is C₃₋₁₀ cycloalkyl; C₄₋₁₀ bicycloalkyl; C₄₋₁₀ heterocyclyl; C₄₋₁₀ heterobicyclyl; aryl having 6 to 10 carbon C atoms; heteroaryl having 5 to 10 ring atoms, wherein T¹ is optionally substituted with one or more R⁹, wherein R⁹ is independently halogen; CN; COOR¹⁰; OR¹⁰; C(O)N(R¹⁰R^{10a}); S(O)₂N(R¹⁰R^{10a}); S(O)N(R¹⁰R^{10a}); S(O)₂R¹⁰; N(R¹⁰)S(O)₂N(R^{10a}R^{10b}); SR¹⁰; N(R¹⁰R^{10a}); OC(O)R¹⁰; N(R¹⁰)C(O)R^{10a}; N(R¹⁰)S(O)₂R^{10a}; N(R¹⁰)S(O)R^{10a}; N(R¹⁰)C(O)N(R^{10a}R^{10b}); N(R¹⁰)C(O)OR^{10a}; OC(O)N(R¹⁰R^{10a}); oxo (═O), where the ring is at least partially saturated; C(O)R¹⁰; C₁₋₆ alkyl; phenyl; C₃₋₇ cycloalkyl; or heterocyclyl, wherein C₁₋₆ alkyl; phenyl; C₃₋₇ cycloalkyl; and heterocyclyl are optionally substituted with one or more halogen, which are the same or different;

R¹⁰, R^{10a} and R^{10b} are independently selected from the group consisting of H, C₁₋₆ alkyl, phenyl, C₃₋₇ cycloalkyl, heteroaryl and heterocyclyl, wherein C₁₋₆ alkyl, phenyl, C₃₋₇ cycloalkyl and heterocyclyl are optionally substituted with one or more halogen, which are the same or different;

R² is hydrogen, C₁₋₄ alkyl, an acetyl group or a urea;

R³ is hydrogen, a hydroxyl, C₁₋₄ alkyl, or amino group; and

X is a bond;

or a pharmaceutically acceptable salt thereof.

Particular preferred compounds for modulating, preferably inhibiting the activity of Mnk1 and/or Mnk2 kinase are the pyrazolopyrimidine compounds EDJ100869, EDJ101424, EDJ101441, EDJ101457, EDJ101458, EDJ101472 and EDJ101496 as described in the co-owned patent application WO 2006/066937 and shown in FIG. 4. The most preferred compound of this class of pyrazolopyrimidine compounds is the compound EDJ100869.

Further, very preferred compounds for modulating, in particular inhibiting the activity of the Mnk1 and/or Mnk2 kinase or variants thereof are thienopyrimidine compounds of the general Formula (II)

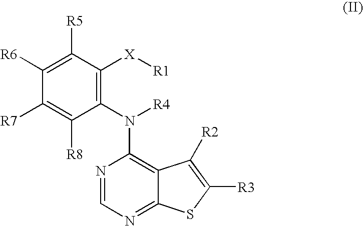

wherein X is O, S, SO₂, CH₂, CHR₁ₐ, CR₁ₐR_{1b}, CH(halogen), C(halogen)₂, C═O, C(O)NR₁ₐ, NH or NR₁ₐ, wherein R₁ₐ and R_{1b} are C₁₋₆ alkyl, C₁₋₆ alkyl C₃₋₁₀ cycloalkyl, C₃₋₁₀ cycloalkyl, C₁₋₆ alkyl 3 to 10 membered heterocycloalkyl comprising at least one heteroatom selected from N, S and O, 3 to 10 membered heterocycloalkyl comprising at least one heteroatom selected from N, S and O, wherein R₁ₐ and R_{1b} are optionally substituted with one or more R₉;

R₁ is hydrogen, C₁₋₆ alkyl, C₁₋₆ alkyl C₃₋₁₀ cycloalkyl, C₃₋₁₀ cycloalkyl, C₁₋₆ alkyl 3 to 10 membered heterocycloalkyl comprising at least one heteroatom selected from N, S and O, 3 to 10 membered heterocycloalkyl comprising at least one heteroatom selected from N, S and O, C₆₋₁₀ aryl, C₁₋₆ alkyl C₆₋₁₀ aryl, C₅₋₁₀ heteroaryl comprising at least one heteroatom selected from N, S and O, C₁₋₆ alkyl C₅₋₁₀ heteroaryl comprising at least one heteroatom selected from N, S and O, wherein R₁ is optionally substituted with one or more R₉;

or if X is NR₁ₐ, CHR₁ₐ, C(O)NR₁ₐ or CR₁ₐR_{1b}, R₁ may form a carbocyclic or heterocyclic ring with R₁ₐ and the N or C atom to which they are attached, which may contain one or more additional heteroatoms selected from N, S and O, which may be substituted with one or more R₉;

R₂ and R₃ are the same or different and are independently selected from hydrogen, C₁₋₆ alkyl, C₁₋₆ alkyl C₃₋₁₀ cycloalkyl, C₃₋₁₀ cycloalkyl, C₆₋₁₀ aryl, C₁₋₆ alkyl C₆₋₁₀ aryl, C₅₋₁₀ heteroaryl comprising at least one heteroatom selected from N, S and O, C₁₋₆ alkyl C₅₋₁₀ heteroaryl comprising at least one heteroatom selected from N, S and O, C₁₋₆ alkyl 3 to 10 membered heterocycloalkyl comprising at least one heteroatom selected from N, S and O, 3 to 10 membered heterocycloalkyl comprising at least one heteroatom selected from N, S and O, or together with the C atoms that they are attached to form a C₃₋₇ cycloalkyl or a 3 to 10 membered heterocycloalkyl group, wherein R₂ and R₃ are optionally substituted with one or more R₉, R₂ may also be R₉ and R₃ may also be R₁₀;

R₄ is hydrogen, C₁₋₄ alkyl, urea, thiourea or acetyl optionally substituted with one or more R₉;

or R₄ may form a 5 or 6 membered heterocyclic ring with R₁:

R₅, R₆, R₇ and R₈ are the same or different and are independently selected from H or R₉;

R₉ is independently halogen; CN; COOR₁₁; OR₁₁; C(O)N(R₁₁R₁₁ₐ); S(O)₂N(R₁₁R₁₁ₐ); S(O)N(R₁₁R₁₁ₐ); S(O)₂R₁₁; N(R₁₁)S(O)₂N(R₁₁ₐR_{11b}); SR₁₁; N(R₁₁R₁₁ₐ); OC(O)R₁₁; N(R₁₁)C(O)R₁₁ₐ; N(R₁₁)S(O)₂R₁₁ₐ; N(R₁₁)S(O)R₁₁ₐ; N(R₁₁)C(O)N(R₁₁ₐR_{11b}); N(R₁₁)C(O)OR₁₁ₐ; OC(O)N(R₁₁R₁₁ₐ); oxo (═O), where the ring is at least partially saturated; C(O)R₁₁; C₁₋₆ alkyl; phenyl; C₃₋₇ cycloalkyl; or heterocyclyl, wherein C₁₋₆ alkyl; phenyl; C₃₋₇ cycloalkyl; and heterocyclyl are optionally substituted with one or more R₁₀;

R₁₀ is independently halogen; CN; OR₁₁; S(O)₂N(R₁₁R₁₁ₐ); S(O)N(R₁₁R₁₁ₐ); S(O)₂R₁₁; N(R₁₁)S(O)₂N(R₁₁ₐR_{11b}); SR₁₁; N(R₁₁R₁₁ₐ); OC(O)R₁₁; N(R₁₁)C(O)R₁₁ₐ; N(R₁₁)S(O)₂R₁₁ₐ; N(R₁₁)S(O)R₁₁ₐ; N(R₁₁)C(O)N(R₁₁ₐR_{11b}); N(R₁₁)C(O)OR₁₁ₐ; OC(O)N(R₁₁R₁₁ₐ); oxo (═O), where the ring is at least partially saturated; C(O)R₁₁; C₁₋₆ alkyl; phenyl; C₃₋₇ cycloalkyl; or heterocyclyl, wherein C₁₋₆ alkyl; phenyl; C₃₋₇ cycloalkyl; and heterocyclyl are optionally substituted with one or more R₉;

R₁₁, R₁₁ₐ, R_{11b} are independently selected from the group consisting of hydrogen, C₁₋₆ alkyl, C₁₋₆ alkyl C₃₋₁₀ cycloalkyl, C₃₋₁₀ cycloalkyl, C₁₋₆ alkyl 3 to 10 membered heterocycloalkyl comprising at least one heteroatom selected from N, S and O, 3 to 10 membered heterocycloalkyl comprising at least one heteroatom selected from N, S and O, C₆₋₁₀ aryl, 5 to 10 membered heteroaryl comprising at least one heteroatom selected from N, S and O, wherein R₁₁, R₁₁ₐ, R_{11b} are optionally substituted with one or more R₉;

or a metabolite, prodrug or a pharmaceutically acceptable salt thereof.

Preferred thienopyrimidine compounds are shown in FIG. 5. A very preferred modulator and in particular an inhibitor of Mnk1 and/or Mnk2 kinase according to the present invention is the thienopyrimidine compound EDJ101401 described in the co-owned patent application PCT/EP2006/005980, which is the 3-ethoxy-4-(5-methyl-thieno[2,3-D]pyrimidine-4-ylamino)-benzamide.

Further preferred inhibitors of the Mnk1 and/or Mnk2 kinase are the thienopyrimidine compounds described in the co-owned patent application EP 06 007 454 filed on 7 Apr. 2006 and the pyrrolopyrimidine compounds of the co-owned European patent application EP 06 014 297 filed on 10 Jul. 2006, which are both herein incorporated by reference.

In a further preferred embodiment, the modulator, in particular inhibitor compound of the invention is a compound of the general Formula (III)

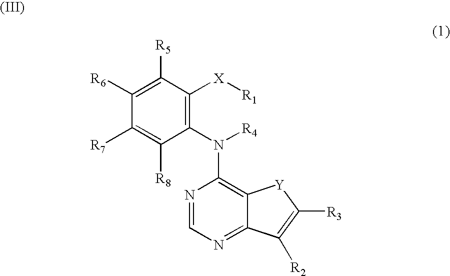

wherein

Y=NH or S;

X is a single bond, O, S, SO₂, CH₂, CHR₁ₐ, CR₁ₐR_{1b}, CH(halogen), C(halogen)₂, C═O, C(O)NR₁ₐ, NH or NR₁ₐ, wherein R₁ₐ and R_{1b} are C₁₋₆ alkyl, C₁₋₆ alkyl C₃₋₁₀ cycloalkyl, C₃₋₁₀ cycloalkyl, C₁₋₆ alkyl 3 to 10 membered heterocycloalkyl comprising at least one heteroatom selected from N, S and O, 3 to 10 membered heterocycloalkyl comprising at least one heteroatom selected from N, S and O, wherein R₁ₐ and R_{1b} are optionally substituted with one or more R₉;

R₁ is hydrogen, C₁₋₆ alkyl, C₁₋₆ alkyl C₃₋₁₀ cycloalkyl, C₃₋₁₀ cycloalkyl, C₁₋₆ alkyl 3 to 10 membered heterocycloalkyl comprising at least one heteroatom selected from N, S and O, 3 to 10 membered heterocycloalkyl comprising at least one heteroatom selected from N, S and O, C₆₋₁₀ aryl, C₁₋₆ alkyl C₆₋₁₀ aryl, C₅₋₁₀ heteroaryl comprising at least one heteroatom selected from N, S and O, C₁₋₆ alkyl C₆₋₁₀ heteroaryl comprising at least one heteroatom selected from N, S and O; wherein R₁ is optionally substituted with one or more R₉;

or if X is NR₁ₐ, CHR₁ₐ, C(O)NR₁ₐ or CR₁ₐR_{1b}, R₁ may form a 5 or 6 membered saturated, unsatured or aromatic carbocyclic or heterocyclic ring with R₁ₐ and the N or C atom to which they are attached, which rings may contain one or more additional heteroatoms selected from N, S and O, which may be substituted with one or more R₉;

R₂ and R₃ are the same or different and are independently selected from hydrogen, C₁₋₆ alkyl, C₁₋₆ alkyl C₃₋₁₀ cycloalkyl, C₃₋₁₀ cycloalkyl, C₆₋₁₀ aryl, C₁₋₆ alkyl C₆₋₁₀ aryl, C₅₋₁₀ heteroaryl comprising at least one heteroatom selected from N, S and O, C₁₋₆ alkyl C₅₋₁₀ heteroaryl comprising at least one heteroatom selected from N, S and O, C₁₋₆ alkyl 3 to 10 membered heterocycloalkyl comprising at least one heteroatom selected from N, S and O, 3 to 10 membered heterocycloalkyl comprising at least one heteroatom selected from N, S and O, or together with the C atoms that they are attached to form a C₃₋₇ cycloalkyl or a 3 to 10 membered heterocycloalkyl group, wherein R₂ and R₃ are optionally substituted with one or more R₉; R₂ may also be R₉ and R₃ may also be R₁₀;

R₄ is hydrogen, C₁₋₄ alkyl, urea, thiourea or acetyl optionally substituted with one or more R₉;

or R₄ may form a 5 or 6 membered saturated, unsatured or aromatic heterocyclic ring with X;

R₅, R₆, R₇ and R₈ are the same or different and are independently selected from hydrogen and R₉;

or R₆ and R₇ may form a 5 or 6 membered saturated, unsatured or aromatic carbocyclic or heterocyclic ring wherein the heterocyclic ring comprises at least one heteroatom selected from N, S and O;

R₉ is independently halogen; CN; COOR₁₁; OR₁₁; C(O)N(R₁₁R₁₁ₐ); S(O)₂N(R₁₁R₁₁ₐ); S(O)N(R₁₁R₁₁ₐ); S(O)₂R₁₁; N(R₁₁)S(O)₂N(R₁₁ₐR_{11b}); N(R₁₁R₁₁ₐ); OC(O)R₁₁; N(R₁₁)C(O)R₁₁ₐ; N(R₁₁)S(O)₂R₁₁ₐ; N(R₁₁)S(O)R₁₁ₐ; N(R₁₁)C(O)N(R₁₁ₐR_{11b}); N(R₁₁)C(O)OR₁₁ₐ; OC(O)N(R₁₁R₁₁ₐ); oxo (═O), where the ring is at least partially saturated; C(O)R₁₁; C₁₋₆ alkyl; phenyl; C₃₋₇ cycloalkyl; or 5 or 6 membered saturated, unsatured or aromatic heterocyclyl comprising at least one heteroatom selected from N, S and O; wherein C₁₋₆ alkyl, phenyl, C₃₋₇ cycloalkyl, and heterocyclyl are optionally substituted with one or more R₁₀;

R₁₀ is independently halogen; CN; OR₁₁; S(O)₂N(R₁₁R₁₁ₐ); S(O)N(R₁₁R₁₁ₐ); S(O)₂R₁₁; N(R₁₁)S(O)₂N(R₁₁ₐR_{11b}); SR₁₁; N(R₁₁R₁₁ₐ); OC(O)R₁₁; N(R₁₁)C(O)R₁₁ₐ; N(R₁₁)S(O)₂R₁₁ₐ; N(R₁₁)S(O)R₁₁ₐ; N(R₁₁)C(O)N(R₁₁ₐR_{11b}); N(R₁₁)C(O)OR₁₁ₐ; OC(O)N(R₁₁R₁₁ₐ); oxo (═O), where the ring is at least partially saturated; C(O)R₁₁; C₁₋₆ alkyl; phenyl; C₃₋₇ cycloalkyl; or heterocyclyl; wherein C₁₋₆ alkyl, phenyl, C₃₋₇ cycloalkyl, and heterocyclyl are optionally substituted with one or more R₉;

R₁₁, R₁₁ₐ, R_{11b} are independently selected from the group consisting of hydrogen, C₁₋₆ alkyl, C₁₋₆ alkyl C₃₋₁₀ cycloalkyl, C₃₋₁₀ cycloalkyl, C₁₋₆ alkyl 3 to 10 membered heterocycloalkyl comprising at least one heteroatom selected from N, S and O, 3 to 10 membered heterocycloalkyl comprising at least one heteroatom selected from N, S and O, C₆₋₁₀ aryl, 5 to 10 membered heteroaryl comprising at least one heteroatom selected from N, S and O, wherein R₁₁, R₁₁ₐ, R_{11b} are optionally substituted with one or more R₉;

or a pharmaceutically acceptable salt thereof.

The use of pharmaceutically acceptable salts of the above-mentioned compounds is also encompassed by the present invention. Pharmaceutically acceptable salts of the compounds of the invention of Formulae (I), (II) and (III) can be formed with numerous organic or inorganic acids and bases and are disclosed in particular in the above-mentioned co-owned patent applications incorporated herein by reference.

Tauopathies are diseases implicating the microtubulae-binding tau. Tau plays a key role in regulating microtubulae dynamics, axional transport and neurite outgrowth, and all these functions of tau are modulated by site-specific phosphorylation. A disruption of normal phosphorylation events results in tau dysfunction and consequently in neurodegenerative diseases. The abnormal tau phosphorylation that occurs in neurodegenerative conditions called taupathies results in a decreased microtubulae binding and in an increased tau-tau interaction, whereby the hyperphosphorylated tau proteins aggregates in paired helico filaments (PHFs) that make up the neurofibrillary tangles (NFTs).

According to the present invention, the tauopathy is selected from a disorder showing a coexistence of tau-containing intracellular neurofibrillary tangles and amyloid-β plaques. For example, neurofibrillary lesions coexist with amyloid-β plaques in Alzheimer's disease, Creutzfeldt-Jakob disease, dementia pugilistica, Down's syndrome, Gerstmann-Sträussler-Sheinker disease, inclusion-body myositis and prion protein cerebral amyloid angiopathy.

On the other hand, the present invention also encompasses taupathies selected from disorders without distinct amyloid-β-containing plaques. Examples of diseases without distinct amyloid-β pathology are frontotemporal dementia (FTD), frontotemporal dementia with parkinsonism linked to chromosome 17 (FTDT-17), Pick's disease, tangle-predominant Alzheimer's disease, corticobasal degeneration, amyotrophic lateral sclerosis/parkinsonism-dementia complex, argyrophilic grain dementia, diffuse neurofibrillary tangles with calcification, Hallevorden-Spatz disease, multiple system atrophy, Niemann-Pick disease Type C, progressive subcortial gliosis, progressive supranuclear palsy and subacute sclerosing panencephalitis.

In a very preferred embodiment, the tauopathy of the present invention refers to Alzheimer's disease.

The modulator of the Mnk1 and/or Mnk2 kinase of the present invention may be used for diagnostic or for therapeutic applications. For the diagnostic application, the modulator may be present in a labelled form, e.g. in a form containing an isotope, e.g. a radioactive isotope or an isotope which may be detected by nucleomagnetic resonance.

The modulator of the Mnk1 and/or Mnk2 kinase of the present invention may be used as an agent for a monotherapy or as an agent for a combination therapy. Hence, the modulator compounds of the present invention may be administrated alone or in combination with at least one further active agent. Preferably, the compounds of the present invention are administered with one further pharmaceutical agent suitable for the alleviation, treatment and/or prevention of a taupathy. In an especially preferred embodiment of the invention, the modulator compounds of the present invention are administered with at least one further agent suitable for the alleviation, treatment and/or prevention of Alzheimer's disease. In particular, further pharmaceutical agents which may be used in combination with the modulator compounds of the present invention are NMDA antagonists such as memantine or acetylcholinesterase inhibitors such as donepezil, rivastigmin and galantamin.

It will be appreciated by the person skilled in the art that the compounds of the invention and the additional therapeutic agents may be formulated in one single dosage form or may be present in separate dosage forms and may either be administered concomittantly, i.e. at the same time, or sequentially.

The agent of the present invention may be formulated in a pharmaceutical composition suitable for the intended method of administration. The compounds of the present invention may be administered by known methods, e.g. by injection, in particular by intravenous, intramuscular, transmucose, subcutaneous or interperintoneal injection and/or by oral, topical, nasal, inhalation, aerosol and/or rectal application etc. The administration may be local or systemic.

For this purpose, the modulator compound of the present invention may be formulated as a pharmaceutical composition. The pharmaceutical composition may contain suitable pharmaceutically acceptable carriers comprising excipients and auxiliaries which facilitate the processing of the active compounds in the preparation. Further details on the techniques for the formulation and administration and on suitable pharmaceutically acceptable carriers and excipients may be found in the latest edition of Remington's Pharmaceutical Science Mack Publishing, Eston, Pa.

The amount of the compounds of the present invention that may be combined with the carriers and/or excipients to formulate a single dosage form will vary on the host treated and the particular mode of administration.

Pharmaceutical compositions suitable for the use in the invention include compositions wherein the active ingredients are contained in an effective amount to achieve the intended purpose. The determination of an effective dose is well within the capability of those skilled in the art. For the purpose of the present invention, a therapeutically effective dosage will generally be from about 1-500 mg/day, preferably from about 10-200 mg/day and most preferably from 10-100 mg/day up to a total dose of about 1 g/day, which may be administered in one or multiple doses.

It will be appreciated, however, that the specific dose level of the compounds of the invention for any particular patient will depend on a variety of factors such as age, sex, body weight, general health condition, diet, individual response of the patient to be treated, time of administration, severity of the disease to be treated, the activity of particular compounds applied, dosage form, mode of application and concommittant medication. The therapeutically effective amount for a given situation will readily be determined by routine experimentation and is within the skills and judgement of the ordinary clinician or physician.

A further object of the present invention is a method of screening of an agent for the diagnosis, alleviation, treatment and/or prevention of a tauopathy comprising the steps
(a) contacting a compound with an at least partially isolated and/or purified Mnk1 and/or Mnk2 kinase,
(b) determining the activity of Mnk1 and/or Mnk2 kinase on phosphorylation of tau protein, and
(c) selecting a compound which reduces the activity of Mnk1 and/or Mnk2 kinase.

The tau protein is preferably a human tau protein or a variant thereof.

In a preferred embodiment of the present invention, the activity of the Mnk1 and/or Mnk2 kinase in steps (b) and (c) of the method of screening outlined above is determined by measuring the phosphorylation of the tau protein on the residues Ser262 and/or Ser356.

A still further object of the present invention is an agent for the diagnosis, alleviation, treatment and/or prevention of a tauopathy comprising the steps of
(a) providing a cell capable of expressing Mnk1 and/or Mnk2 kinase or/and providing a brain extract containing Mnk1 and/or Mnk2 kinase,
(b) contacting a compound with the cell and/or the brain extract,
(c) determining the amount and/or the activity of Mnk1 and/or Mnk2 kinase and
(d) selecting a compound which reduces the amount or/and the activity of Mnk1 and/or Mnk2 kinase.

The activity of the Mnk1 and Mnk2 kinase in step (c) and step (d) of this further method of screening is determined by the degree of phosphorylation of the tau protein. The tau protein is preferably a human tau protein.

According to a very preferred embodiment of the invention, the activity of the Mnk1 and/or Mnk2 kinase in steps (c) and (d) of the method of screening outlined above is determined by the measuring of phosphorylation of the tau protein on the residues Ser262 and/or Ser356.

Moreover, the present invention refers to a method for the diagnosis, alleviation, treatment and/or prevention of a tauopathy comprising administering to a subject in need thereof a pharmaceutically effective amount of a modulator of Mnk1 and/or Mnk2 kinase.

### BRIEF DESCRIPTION OF THE FIGURES

FIG. 1: Western blot analysis shows the phosphorylation of Ser262 and Ser356 in human Tau by Mnk1 and Mnk2 in vitro. Erk2 was used to activate Mnk and is contained in Mnk reactions. Erk2 alone does not phosphorylate Ser262 and Ser356 in human Tau. PKA phosphorylation was used as positive control. Kinase reactions were incubated in presence of 0.5 μM human Tau and 500 μM ATP for 120 min at 37° C. Total Tau served as the loading control.

FIG. 2: The specific Mnk inhibitor EDJ101401 inhibits the in vitro phosphorylation of Ser262 (A) and Ser356 (B) in human Tau by Mnk1 and Mnk2 in a dose dependent manner. Erk2 (5 nM) was used to activate Mnk and is contained in Mnk reactions. Erk2 does not phosphorylate Ser262 and Ser356 in human Tau. PKA phosphorylation was used as positive control. Kinase reactions were incubated in presence of 0.5 μM human Tau and 500 μM ATP for 120 min at 37° C. Total Tau served as the loading control.

FIG. 3: The specific Mnk inhibitor EDJ100869 inhibits the in vitro phosphorylation of Ser262 (A) and Ser356 (B) in human Tau by Mnk2 in a dose dependent manner. PKA phosphorylation was used as positive control. Kinase reactions were incubated in presence of 0.5 μM human Tau and 500 μM ATP for 120 min at 37° C. Total Tau served as the loading control.

FIG. 4 shows preferred pyrazolopyrimidine compounds as inhibitors of Mnk1 and/or Mnk2.

FIG. 5 shows preferred thienopyrimidine compounds as inhibitors of Mnk1 and/or Mnk2.

### EXAMPLES

### 1. In Vitro Phosphorylation of Human Tau by Mnk Kinases

Kinase reaction: The kinase reactions were carried out in a total volume of 30 μl in reaction buffer (20 mM HEPES/KOH pH 7.4, 10 mM MgCl₂, 2 mM DTT, 0.1% Pluronic F127, 0.01% BSA) containing 500 μM ATP, 0.5 μM human recombinant Tau (USBiological, T1040-10), inhibitors and kinases as indicated in figures. Human Mnk1-GST and Mnk2-GST was expressed in E. coli and purifies by Äkta Explorer 100 (Amersham) and pre-activated by human recombinant Erk2 (DeveloGen). Aliquots were stored at −80° C. up to use. Recombinant human PKA (Calbiochem, 539482) was used as positive control. All components of reactions were pre-diluted in reaction buffer. The reactions were incubated for 120 min at 30° C. and stopped by addition of 20 mM EDTA. Samples were analysed by Immunoblotting.

Immunoblotting: Laemmli sample buffer (Biorad) was added to the stopped kinase reactions, subjected to SDS-PAGE and electroblotted on a nitrocellulose membrane (Schleicher & Schüll). The transferred membrane was incubated with NET-G buffer (50 mM Tris/HCl pH 7.5, 5 mM EDTA, 150 mM NaCl, 0.05% Triton X-100, 0.25% (w/v) gelatine) containing 1/1000 (v/v) anti Tau (phospho S262) or anti Tau (phospho S356) antibodies (Abcam, ab4856 and ab4857) over night at 4° C. After washing with NET-G buffer the membrane was treated with NET-G buffer containing 1/5000 (v/v) HRP-conjugated anti rabbit IgG antibody (Pierce) for 2 hours at room temperature.

For loading control membrane was stripped with stripping buffer (200 mM glycine/HCl pH 2.2, 0.1% SDS, 0.1% Tween 20) for 3 hours at room temperature. The membrane was then treated with NET-G buffer containing 1/4000 (v/v) anti Tau antibody (Abcam, ab19326). After washing with NET-G buffer the membrane was incubated with NET-G buffer containing 1/5000 (v/v) HRP-conjugated anti goat IgG antibody (DakoCytomation). Signals were detected with chemiluminescence using ECL Super Signal West Dura kit (Pierce).

### 2. Results

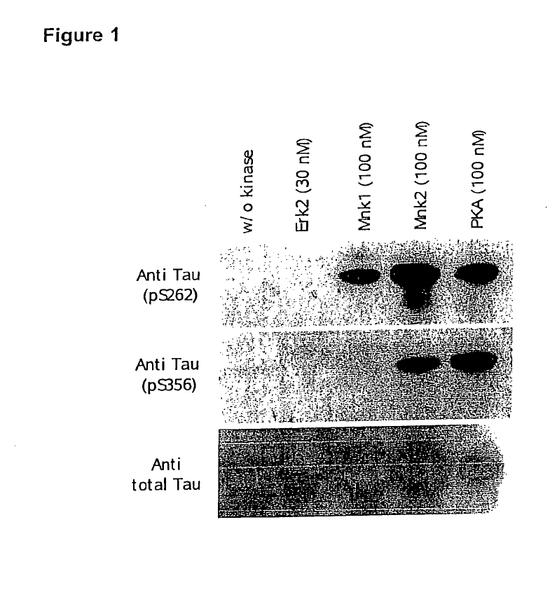

FIG. 1 shows that the tau phosphorylation is effected by Mnk1 and Mnk2 at the phosphorylation sites Ser262 and Ser356, corresponding to the relevant sites on the microtubule binding region. These phosphorylation sites are responsible for the development of tauopathies and especially Alzheimer's disease.

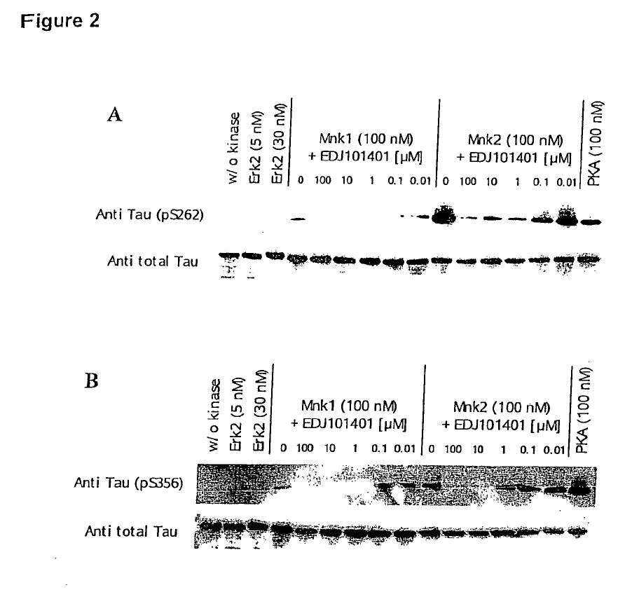

FIG. 2 shows the dose-dependent inhibition of the Mnk1 and Mnk2-dependent tau phosphorylation by thieonopyrimidine substances EDJ101401.

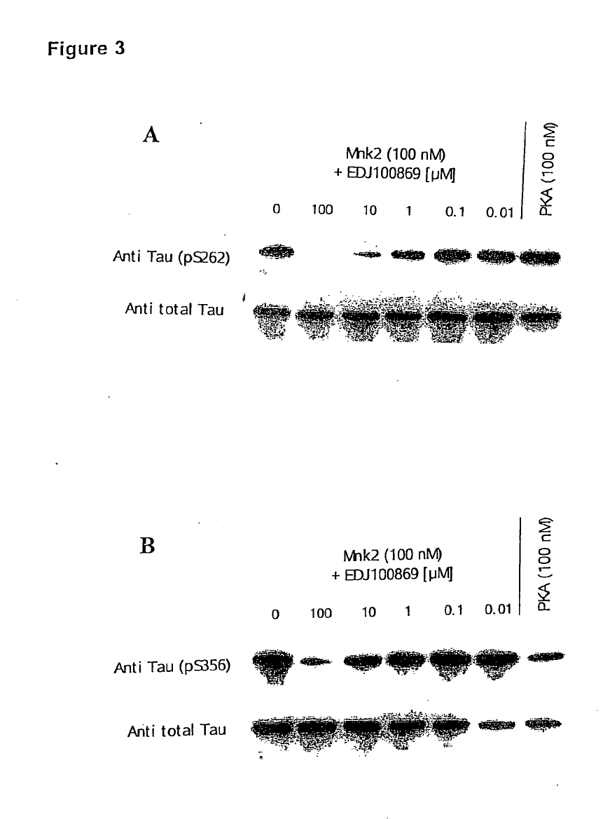

FIG. 3 shows the dosage-dependent inhibition of Mnk1 and Mnk2-dependent tau phosphorylation by the pyrazolopyrimidine compound EDJ100869.

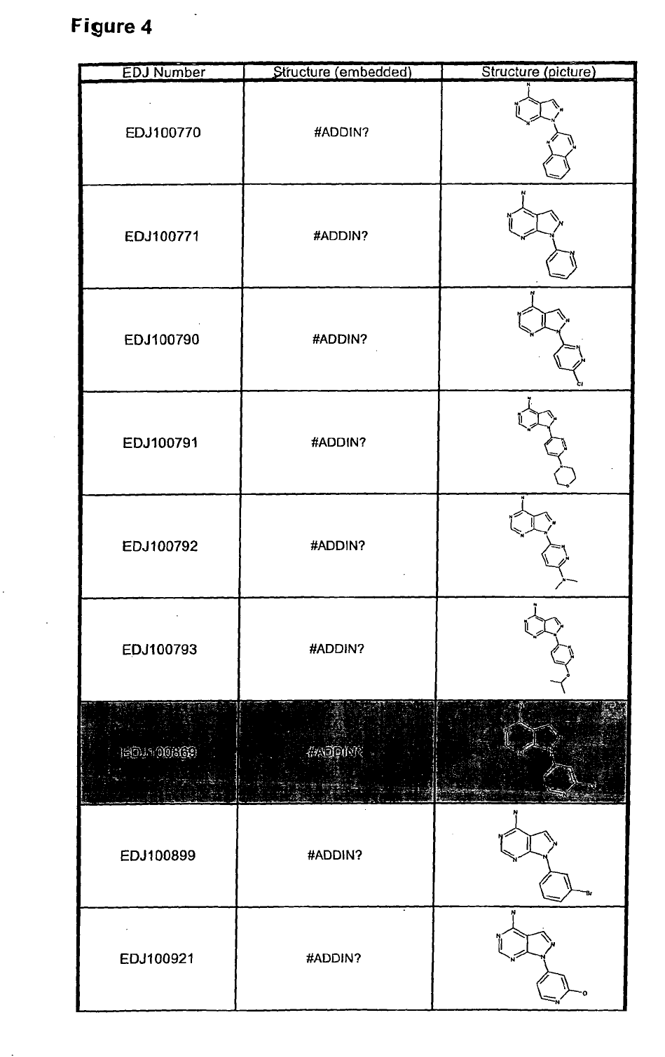

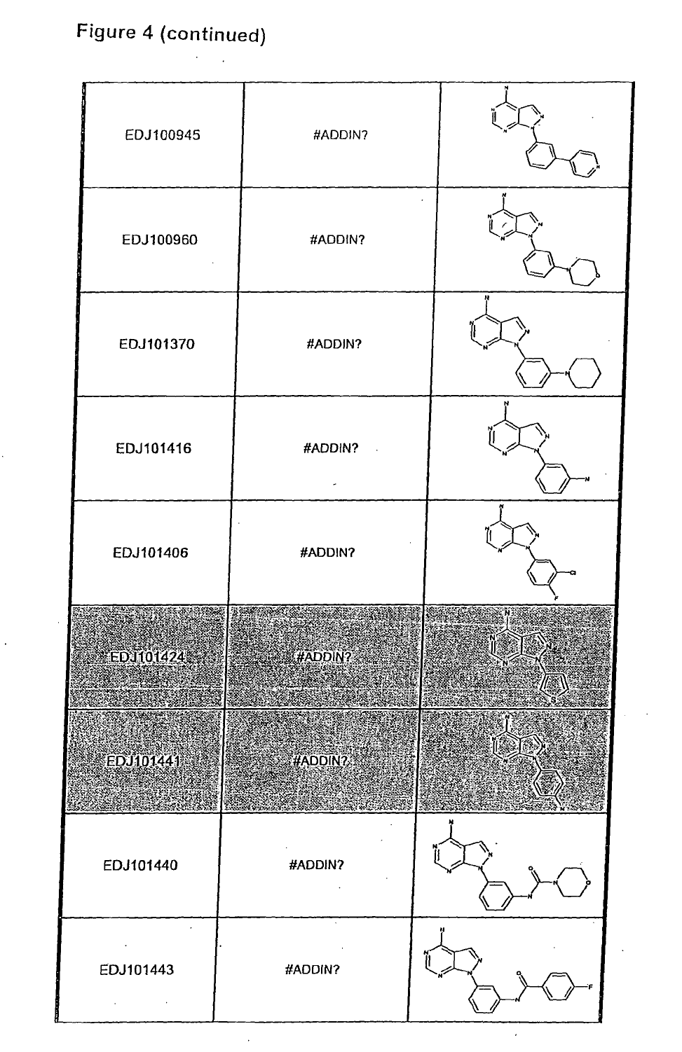

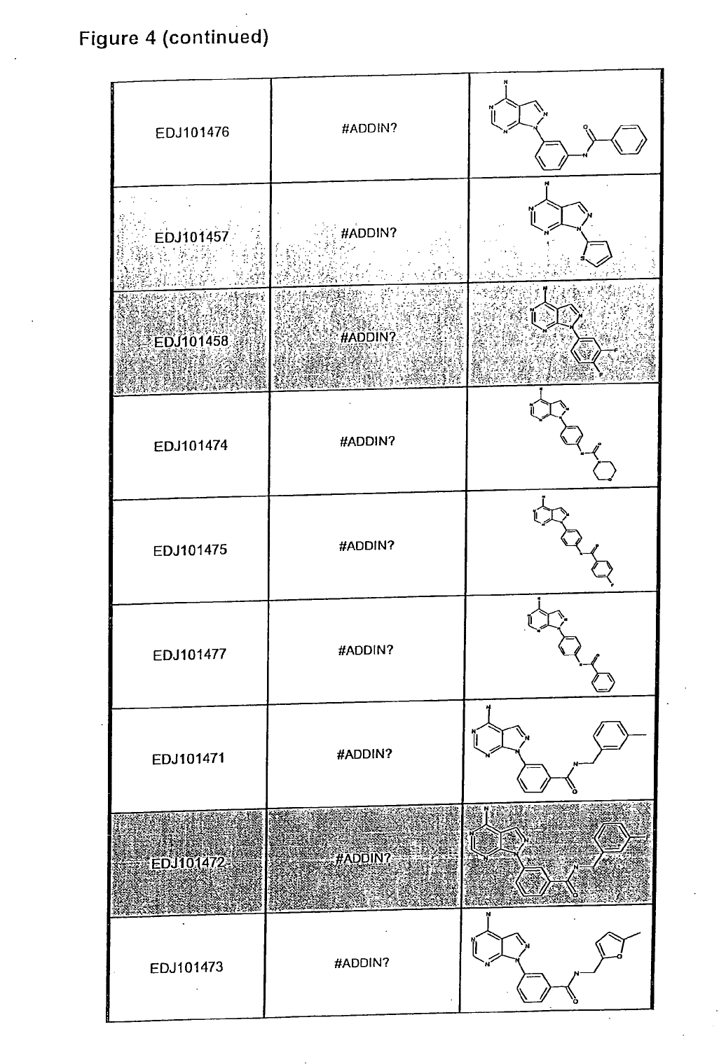

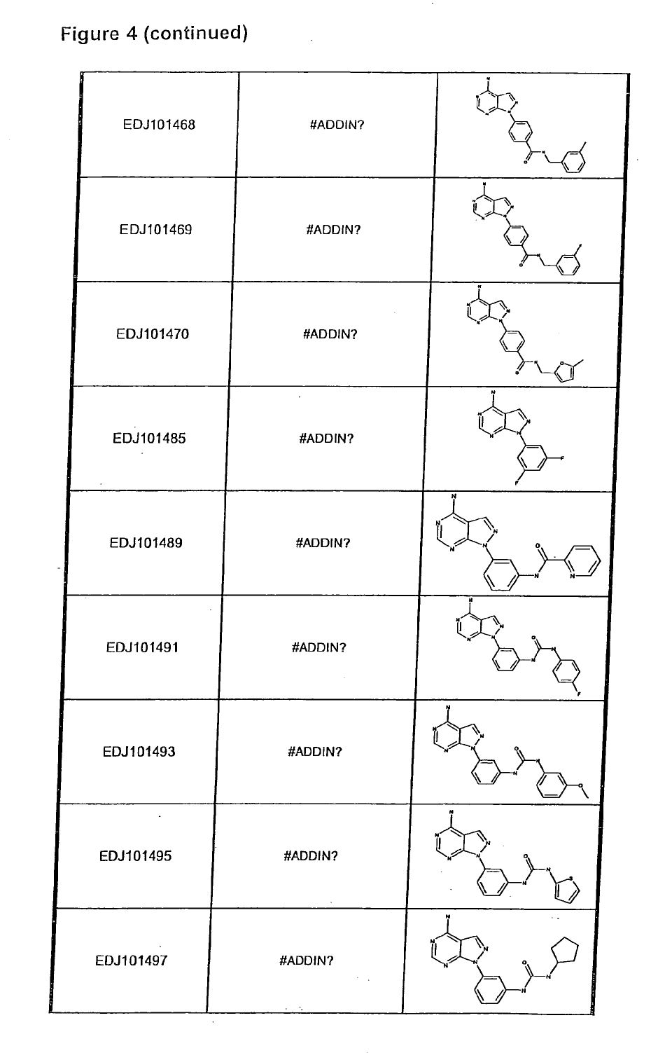

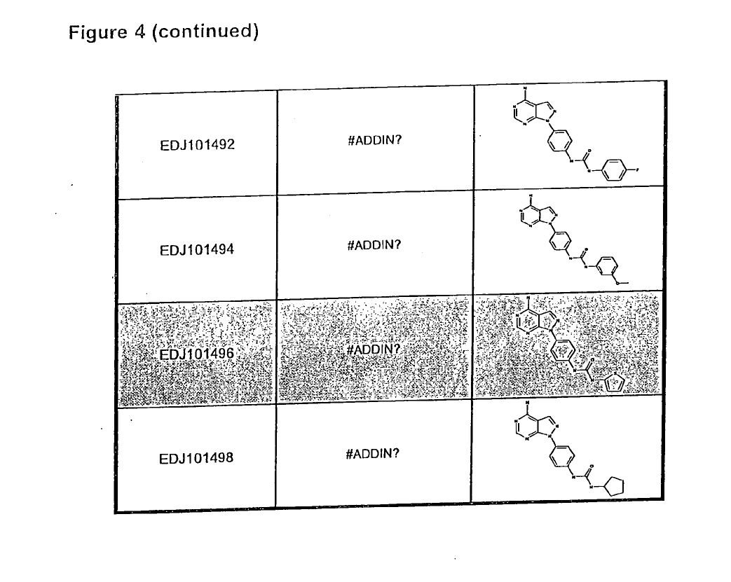

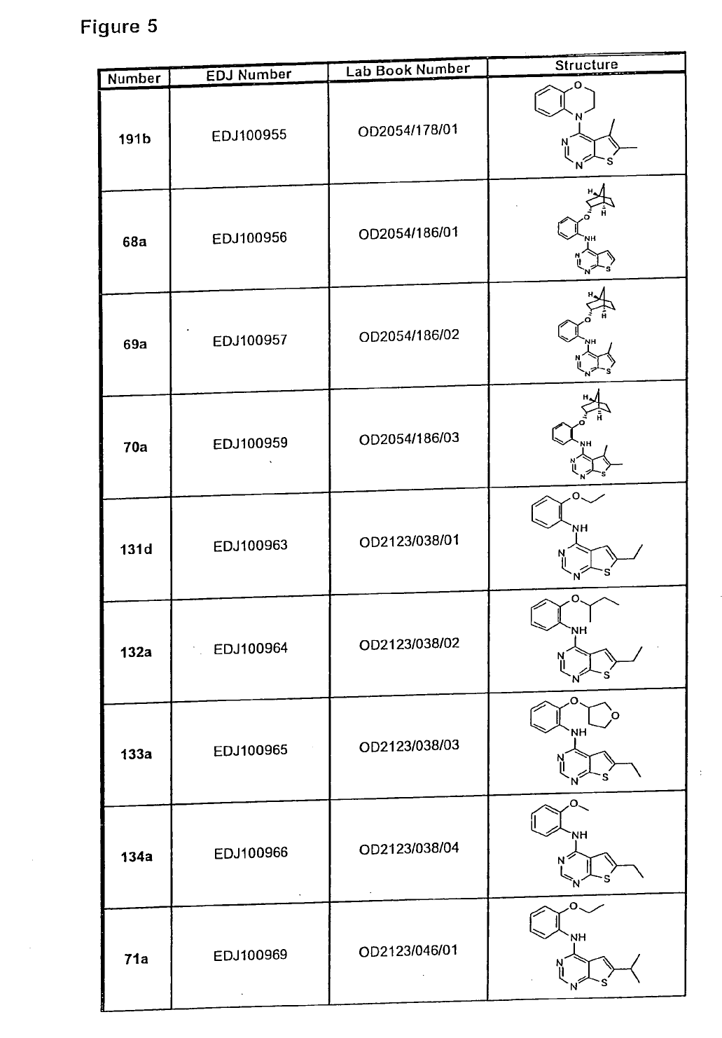

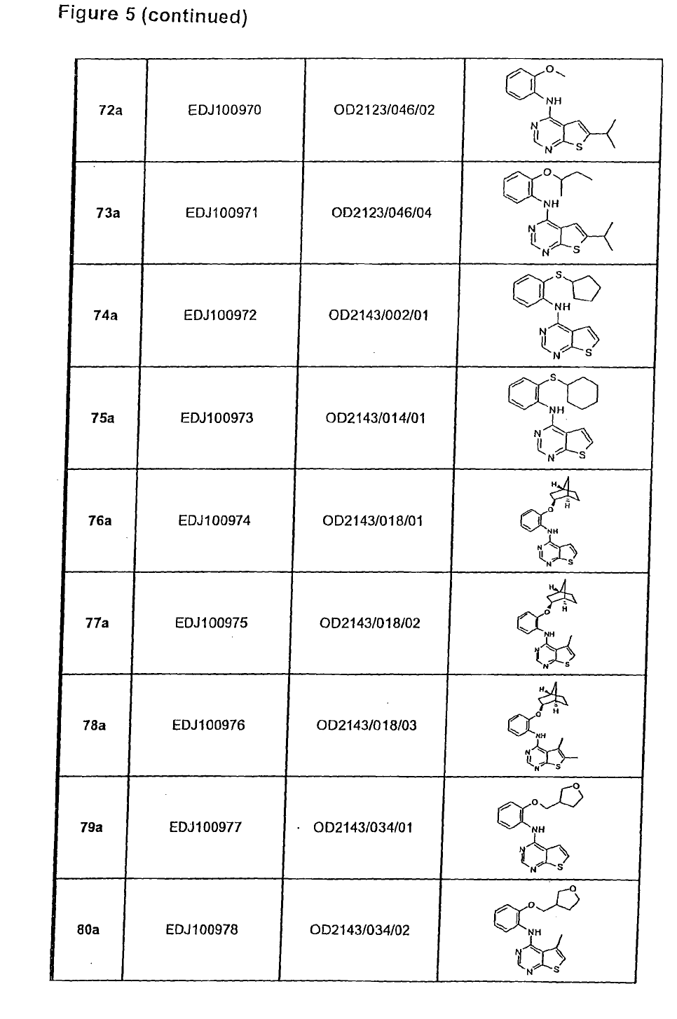

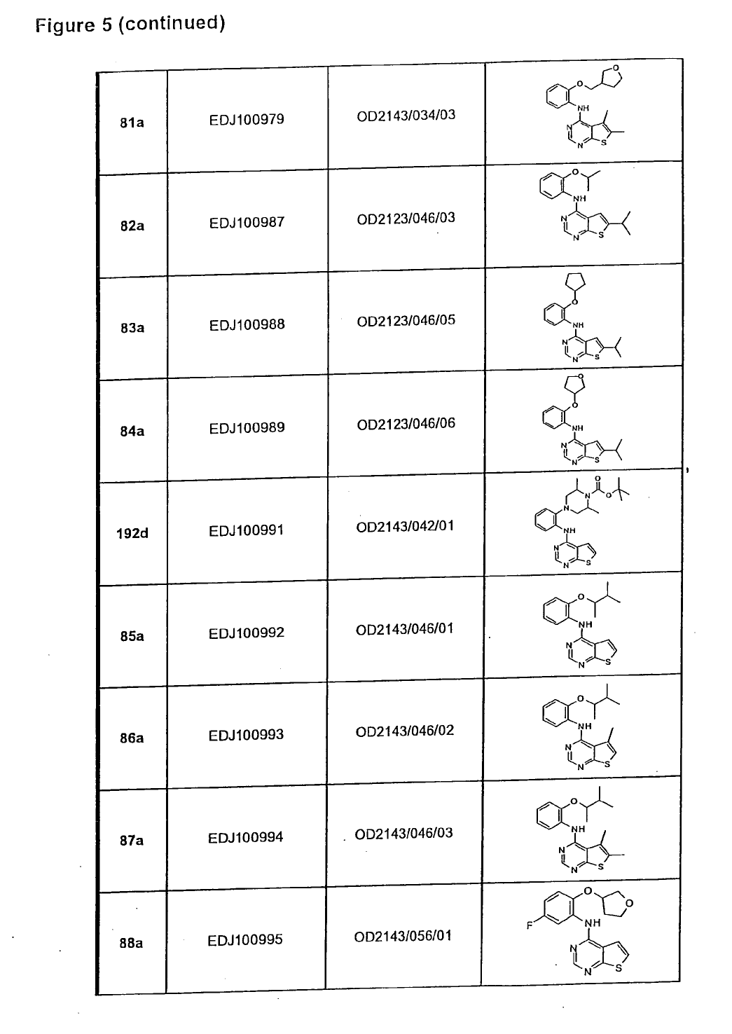

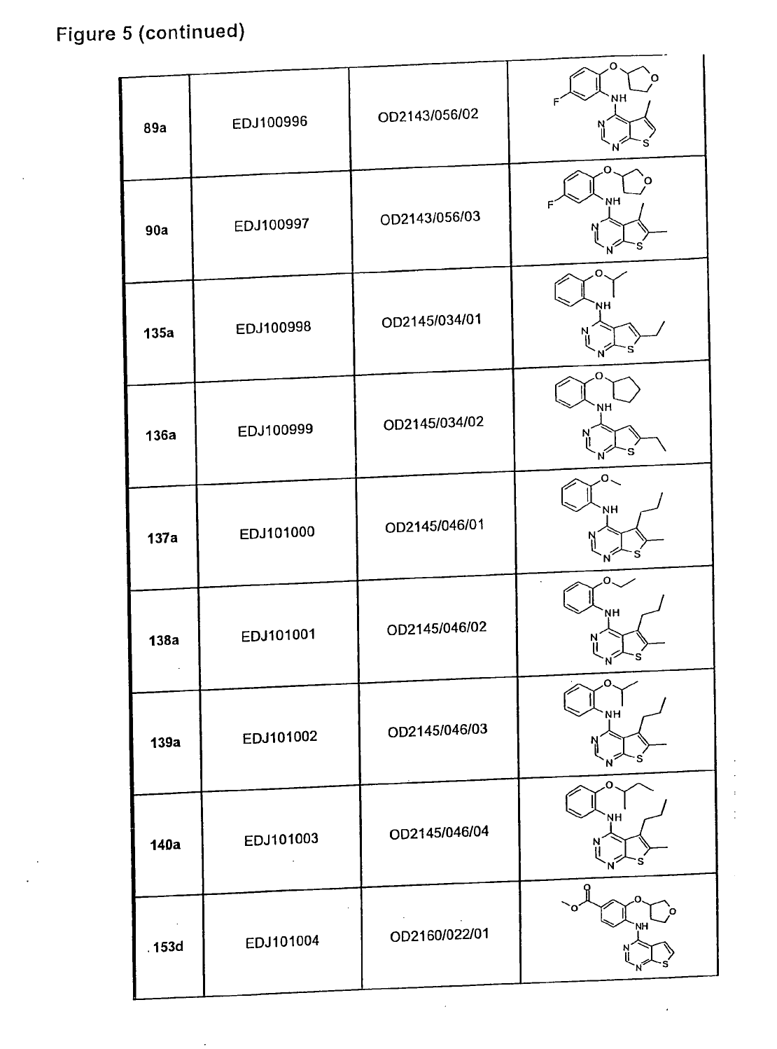

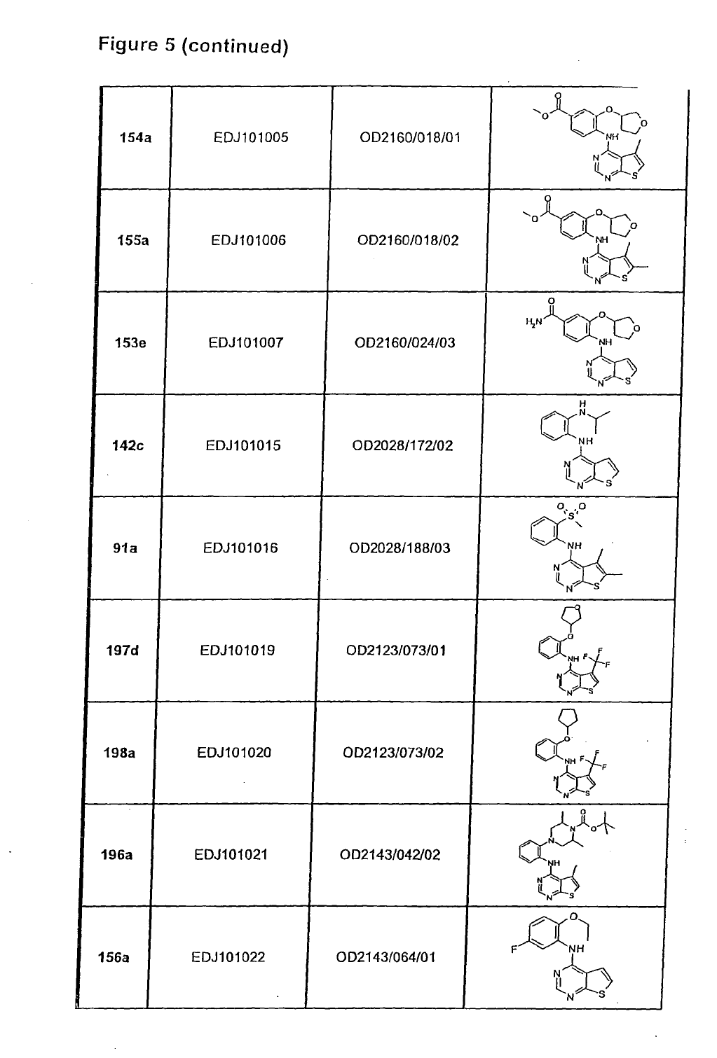

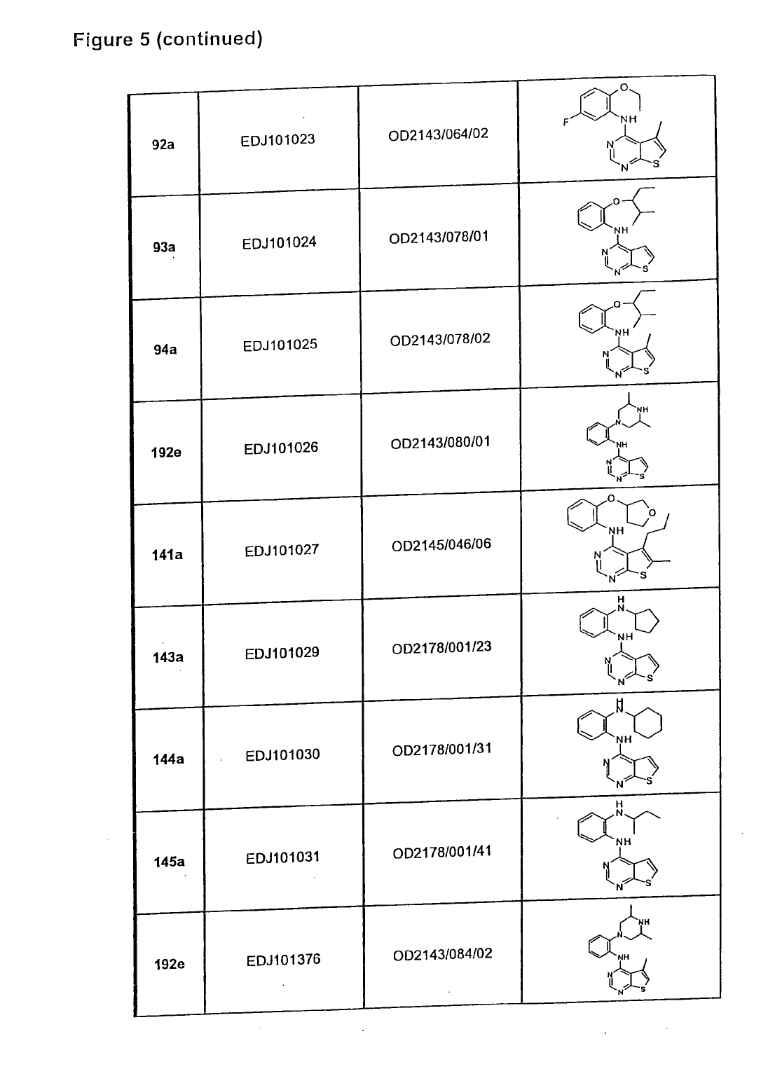

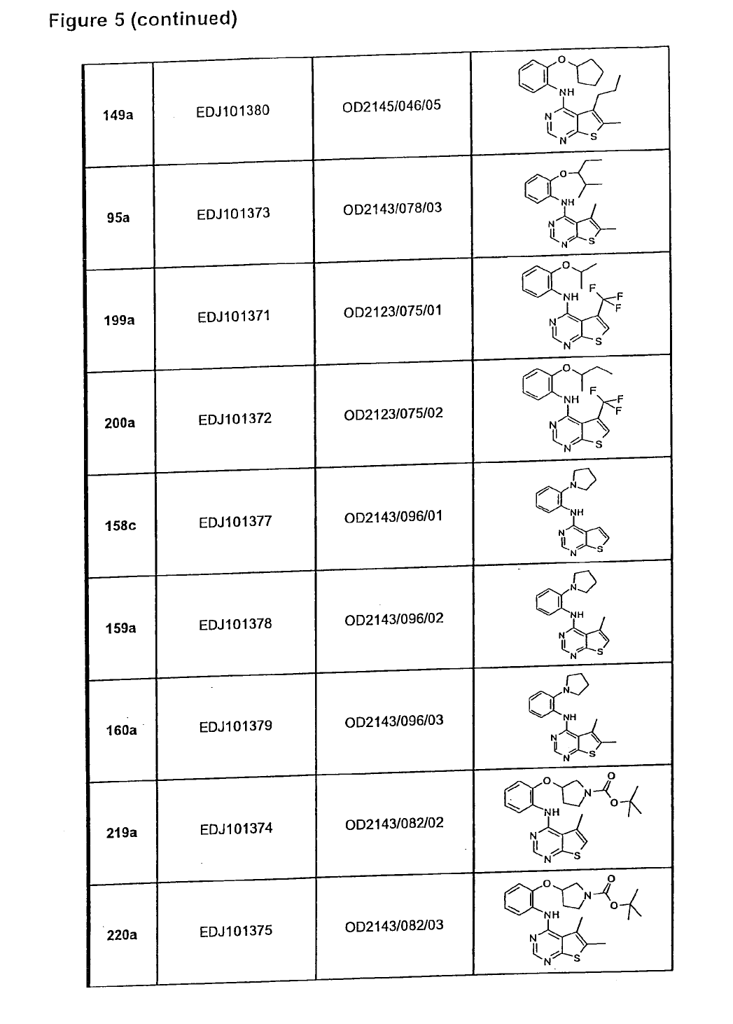

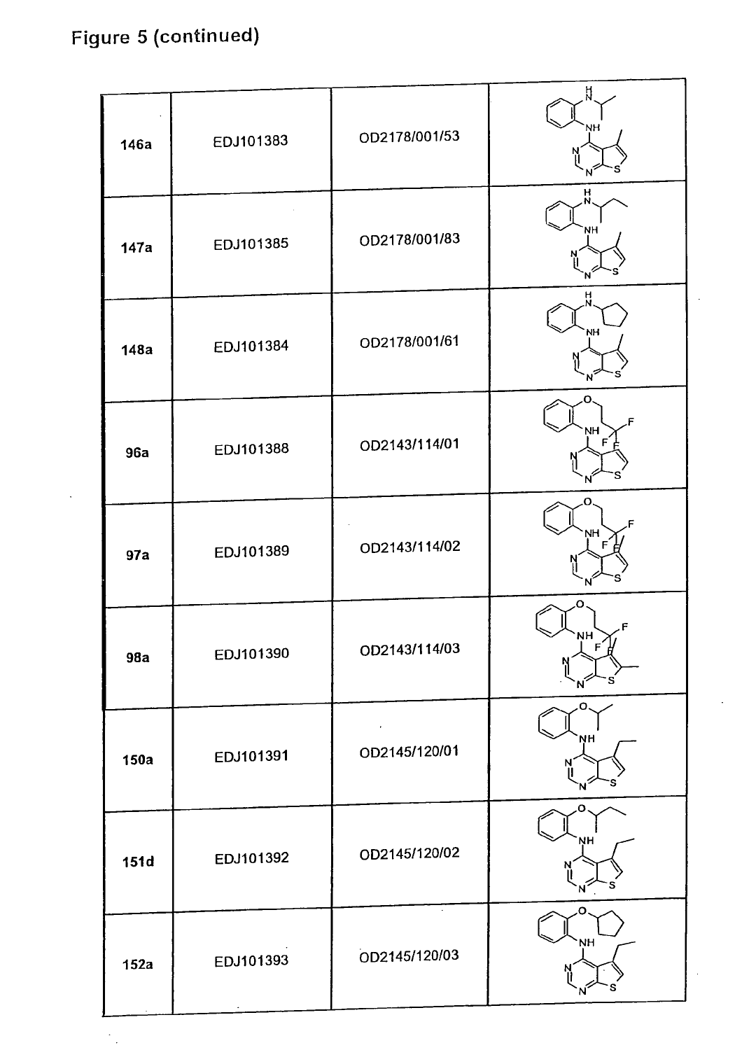

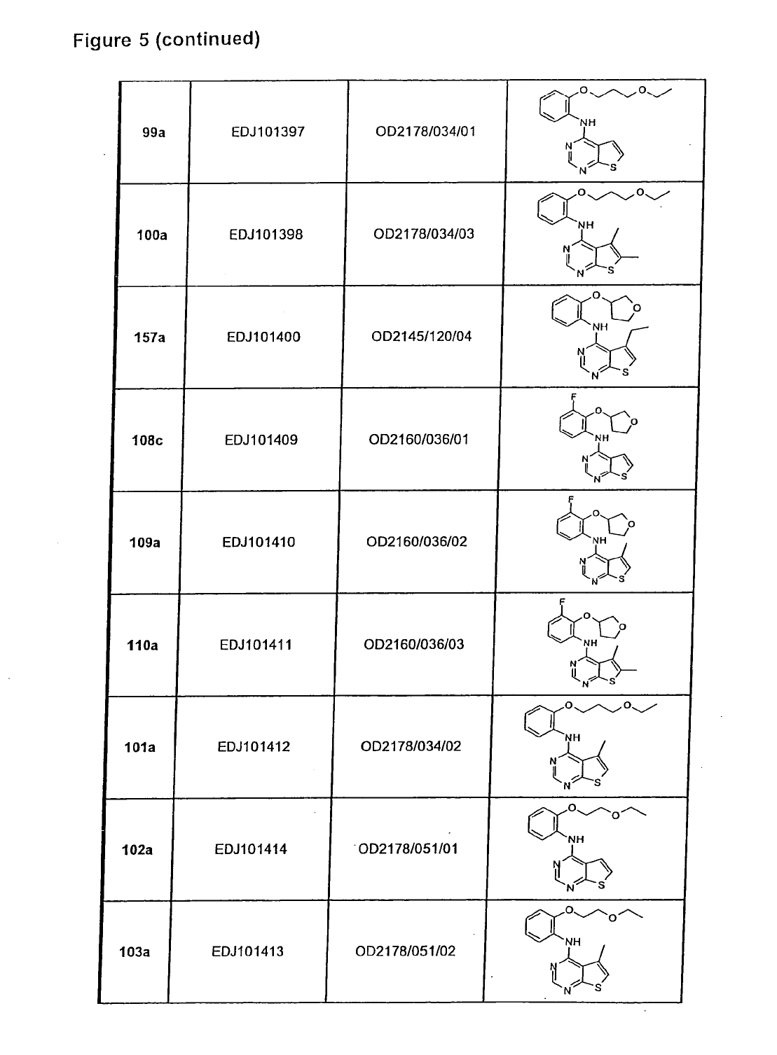

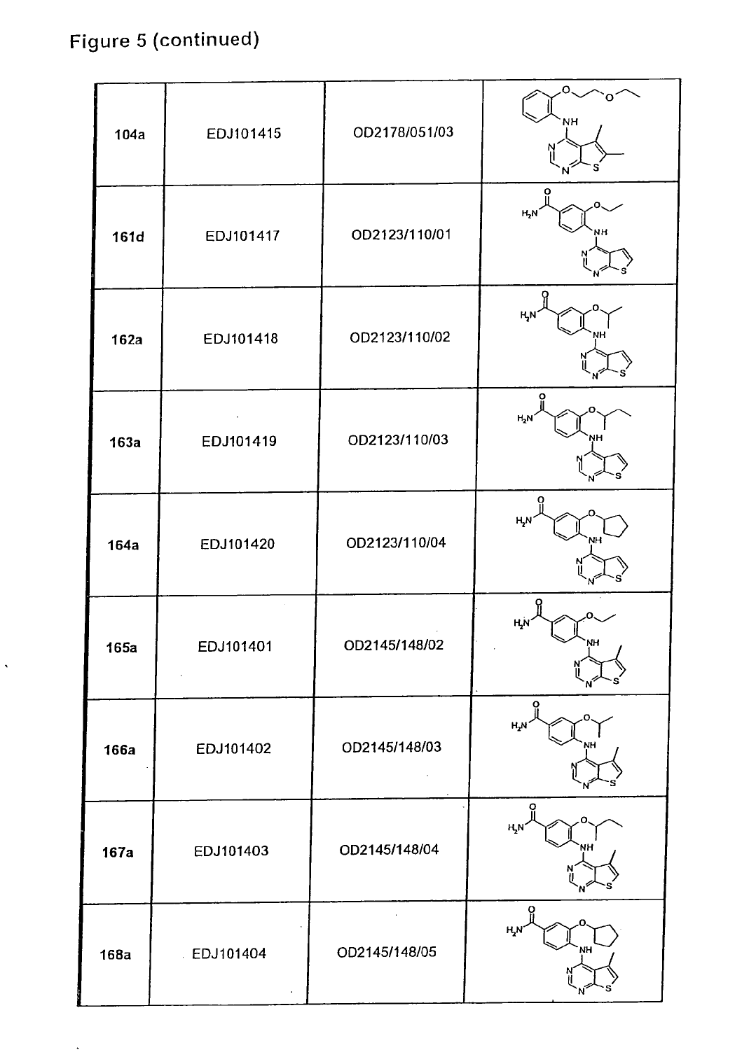

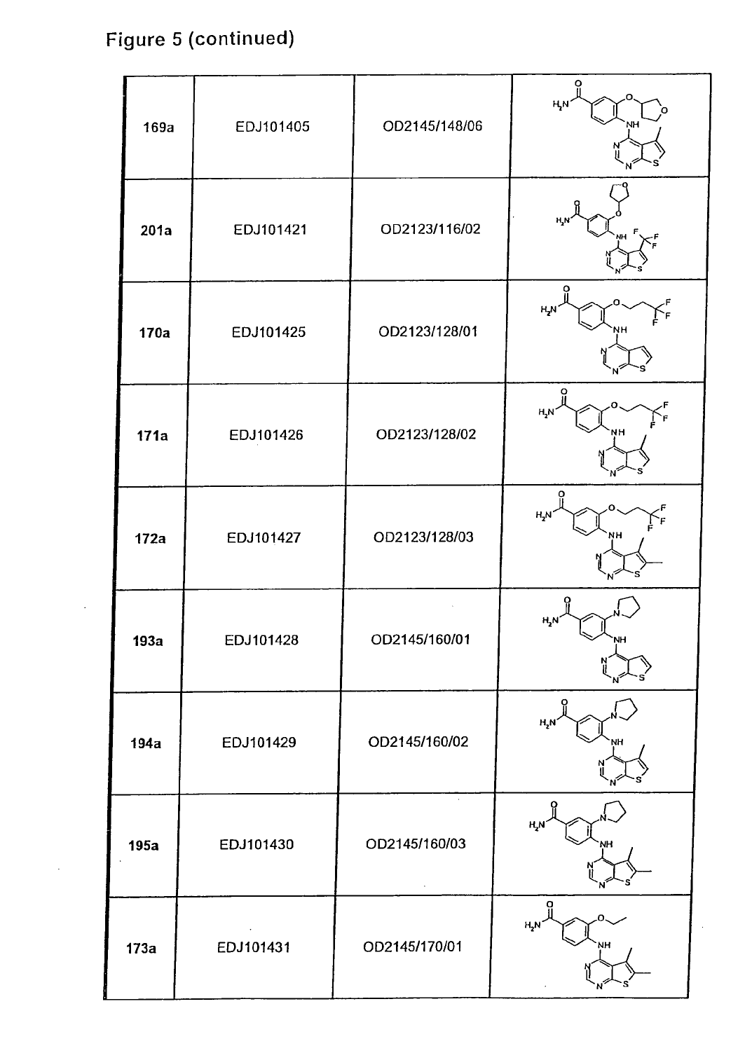

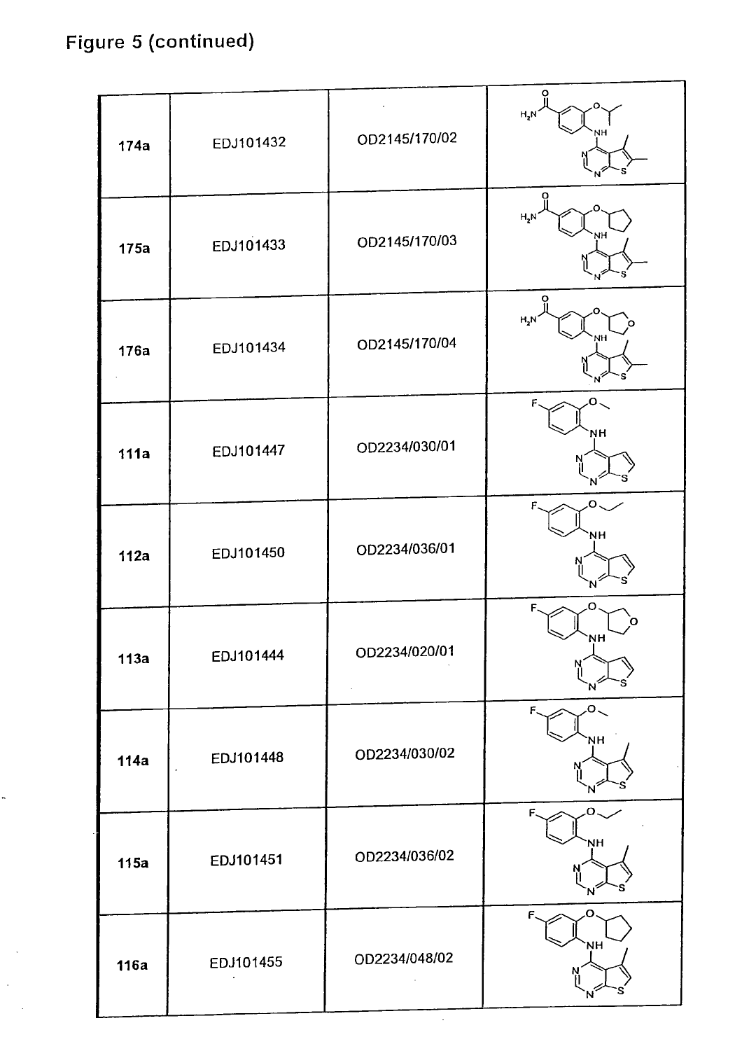

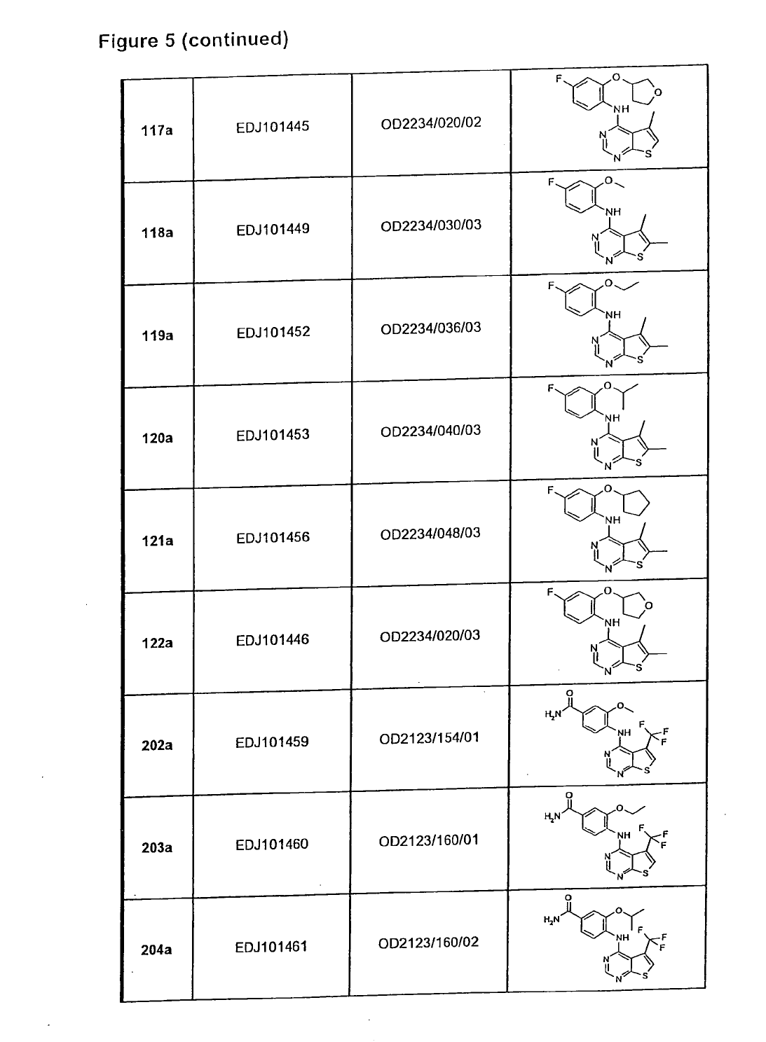

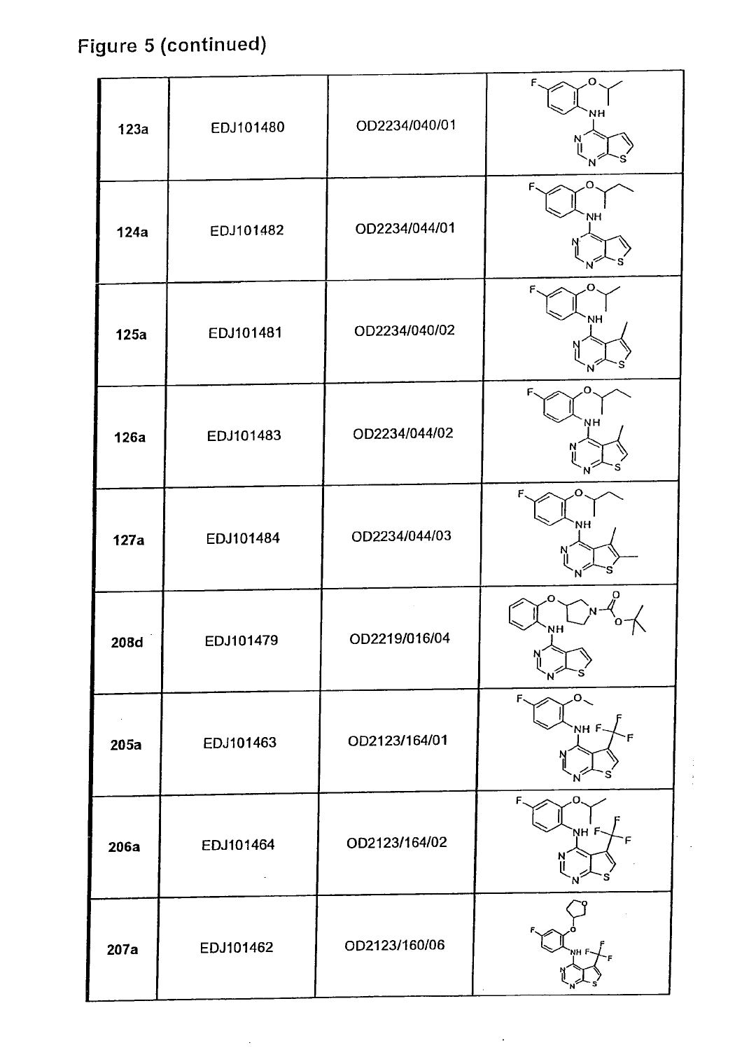

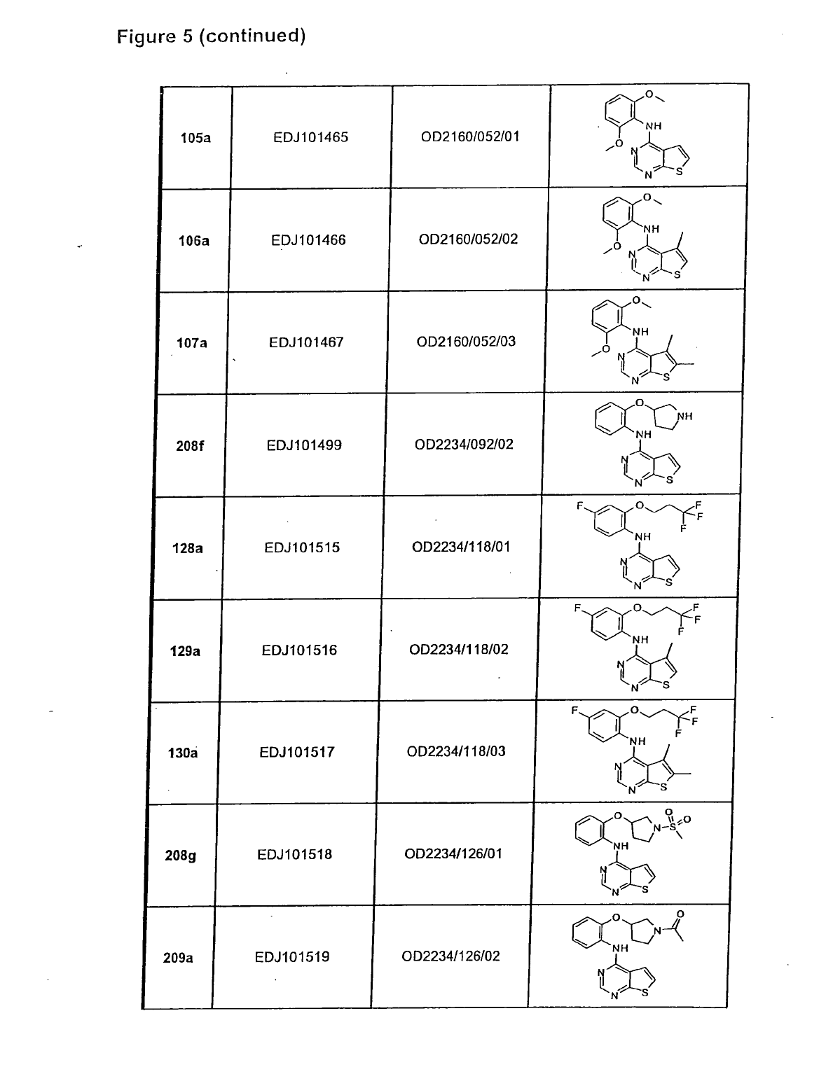

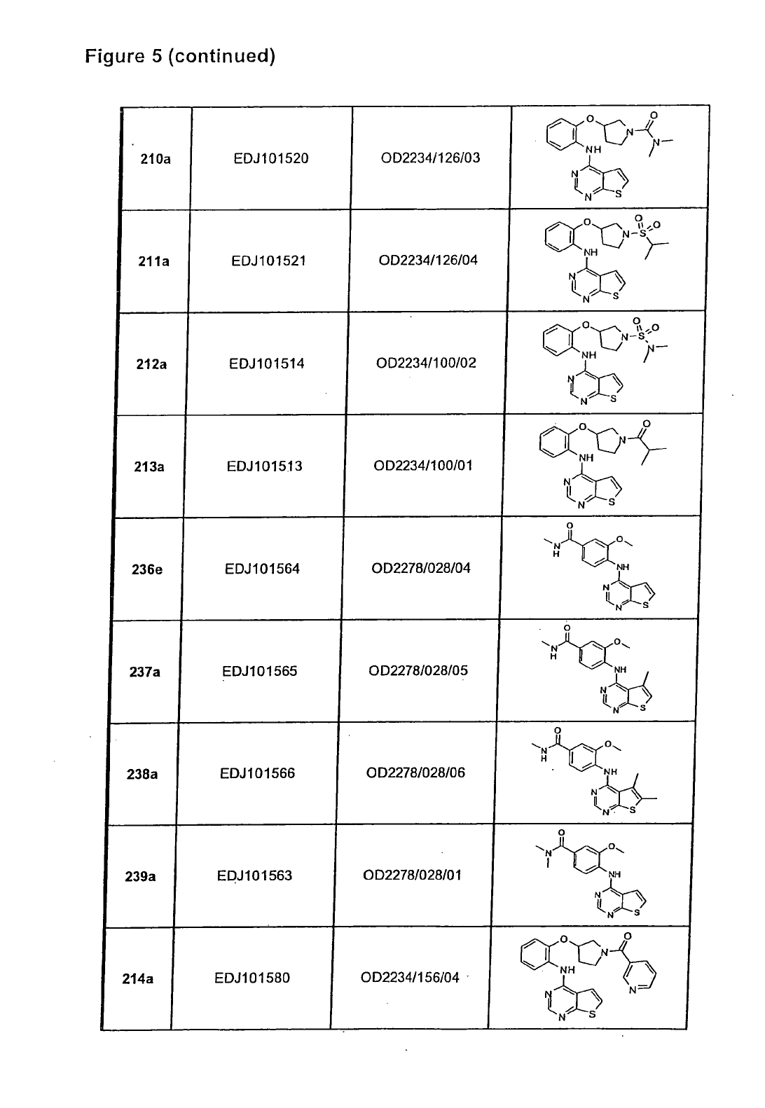

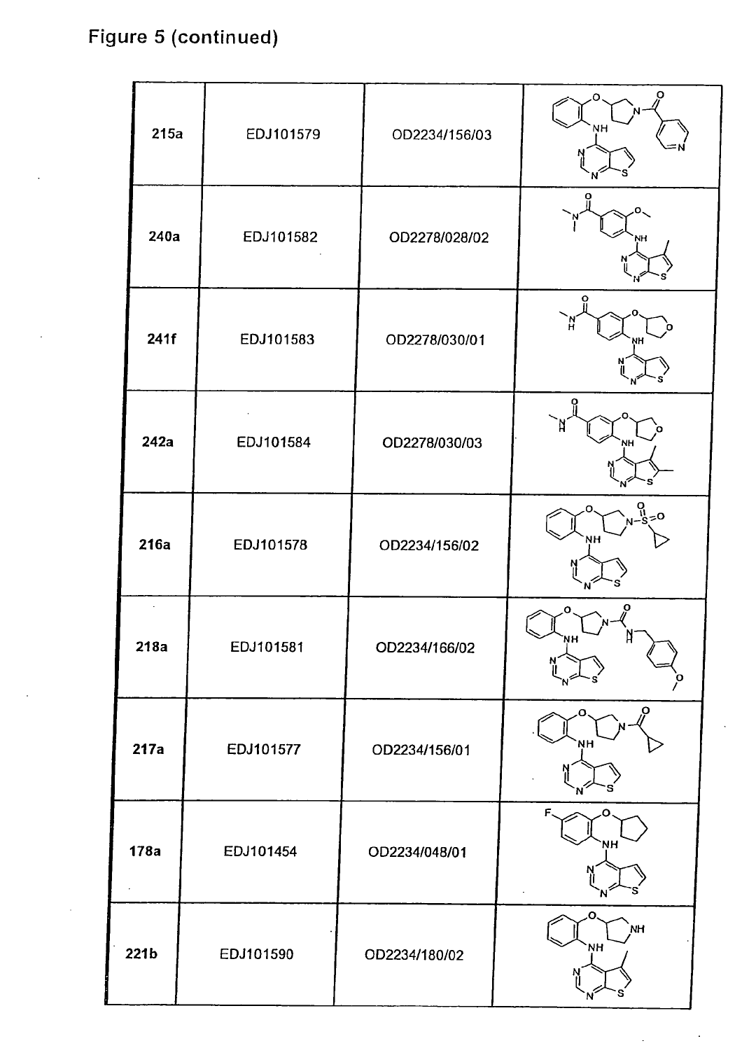

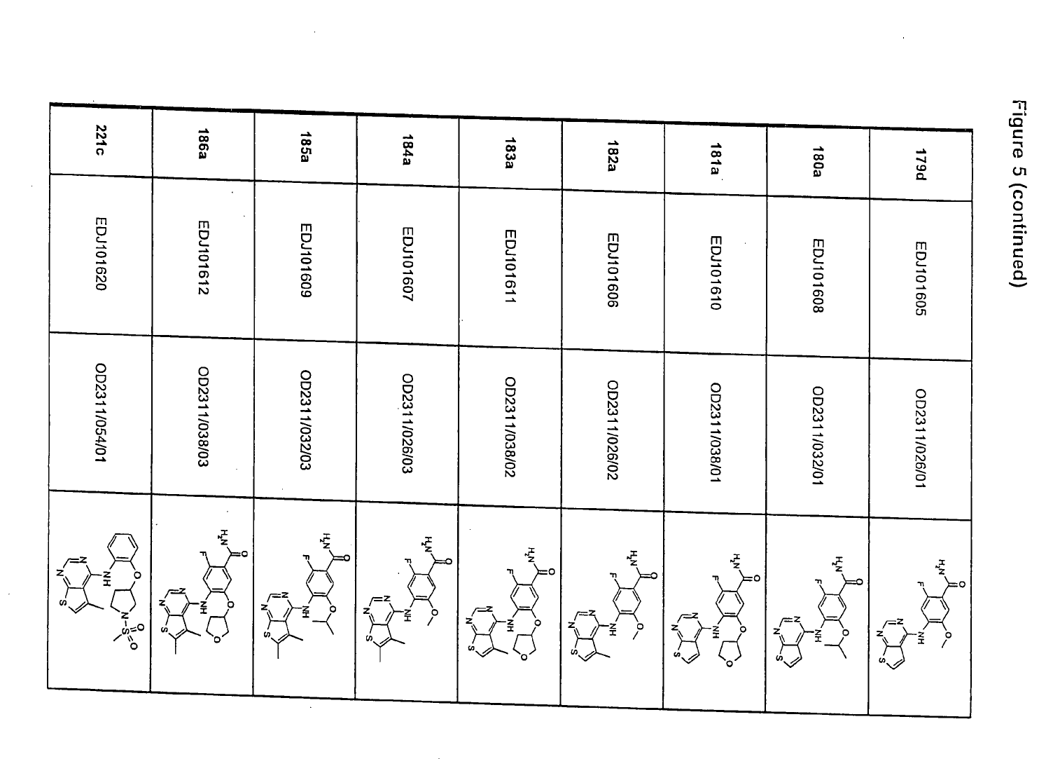

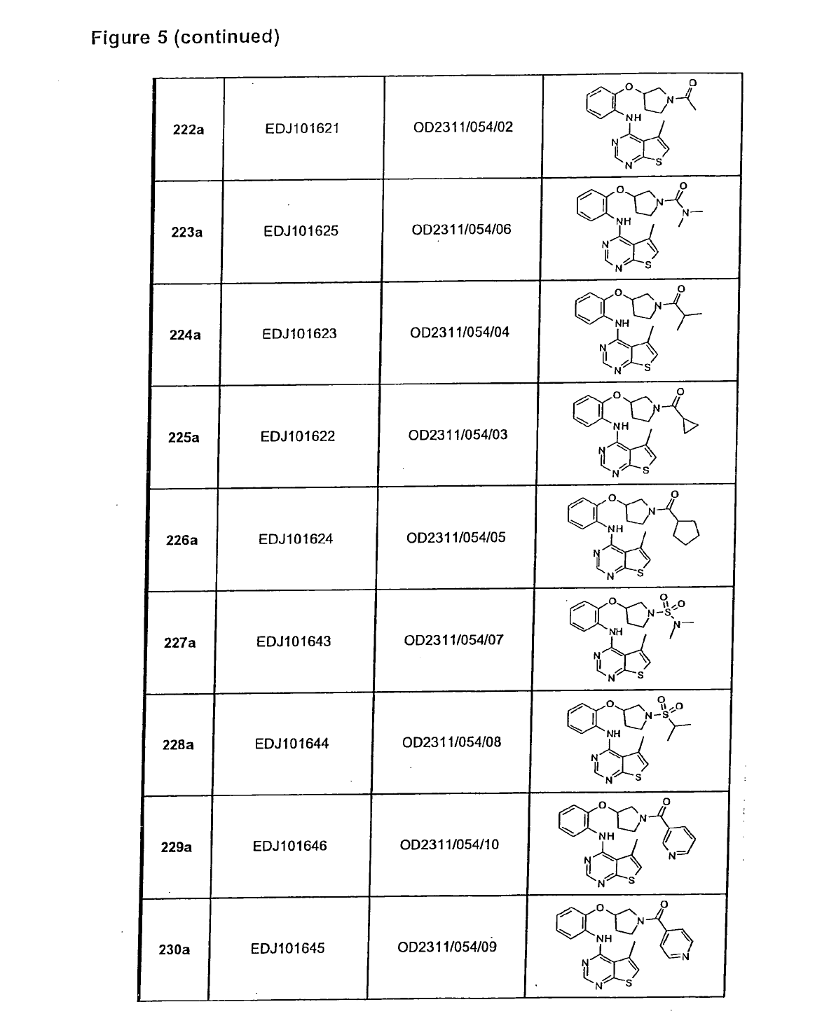

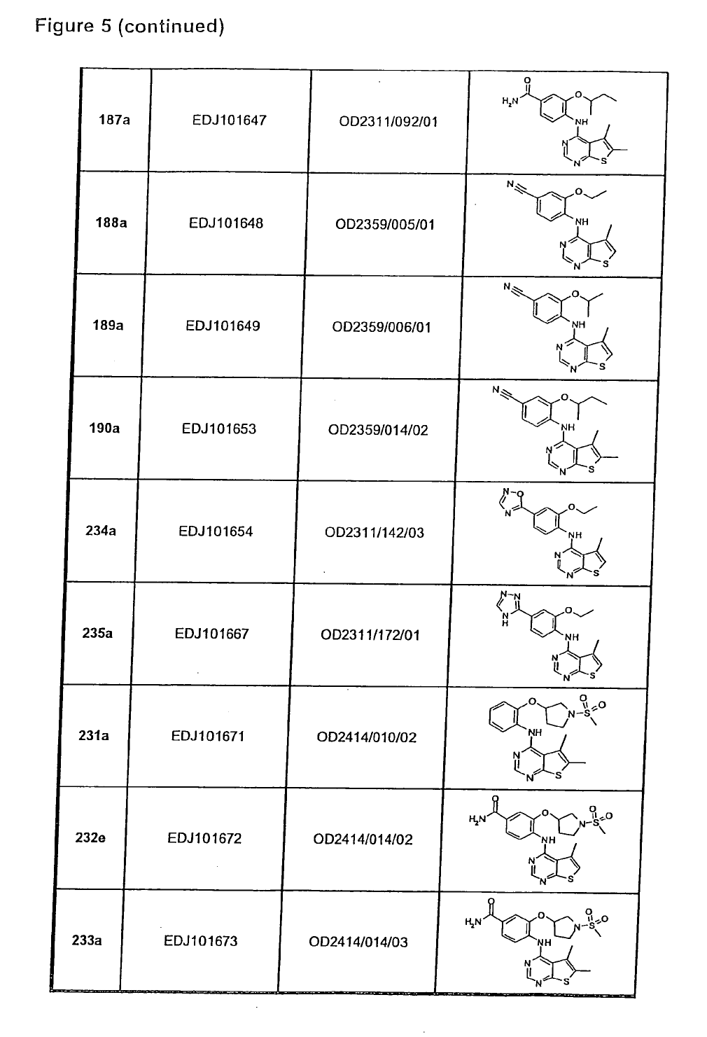

FIGS. 4 and 5 show further preferred examples of Mnk inhibitors.

### REFERENCES

Augustinack, J. C., Schneider, A., Mandelkow, E. M., and Hyman, B. T. (2002). Specific tau phosphorylation sites correlate with severity of neuronal cytopathology in Alzheimer's disease. Acta Neuropathol (Berl) 103, 26-35.
Biernat, J., Gustke, N., Drewes, G., Mandelkow, E. M., and Mandelkow, E. (1993). Phosphorylation of Ser262 strongly reduces binding of tau to microtubules: distinction between PHF-like immunoreactivity and microtubule binding. Neuron 11, 153-163.
Buxade, M., Parra, J. L., Rousseau, S., Shpiro, N., Marquez, R., Morrice, N., Bain, J., Espel, E., and Proud, C. G. (2005). The Mnks are novel components in the control of TNF alpha biosynthesis and phosphorylate and regulate hnRNP A1. Immunity 23, 177-189.
Drewes, G., Trinczek, B., Illenberger, S., Biernat, J., Schmitt-Ulms, G., Meyer, H. E., Mandelkow, E. M., and Mandelkow, E. (1995). Microtubule-associated protein/microtubule affinity-regulating kinase (p110mark). A novel protein kinase that regulates tau-microtubule interactions and dynamic instability by phosphorylation at the Alzheimer-specific site serine 262. J Biol Chem 270, 7679-7688.
Eikelenboom, P., Veerhuis, R., Scheper, W., Rozemuller, A. J., van Gool, W. A., and Hoozemans, J. J. (2006). The significance of neuroinflammation in understanding Alzheimer's disease. J Neural Transm 113, 1685-1695.
Goedert, M., and Spillantini, M. (2006). A century of Alzheimer's disease. Science 314, 777-780.
Gomez-Isla, T., Hollister, R., West, H., Mui, S., Growdon, J. H., Petersen, R. C., Parisi, J. E., and Hyman, B. T. (1997). Neuronal loss correlates with but exceeds neurofibrillary tangles in Alzheimer's disease. Ann Neurol 41, 17-24.
Hasegawa, M., Morishima-Kawashima, M., Takio, K., Suzuki, M., Titani, K., and Ihara, Y. (1992). Protein sequence and mass spectrometric analyses of tau in the Alzheimer's disease brain. J Biol Chem 267, 17047-17054.
Hasegawa, M., Smith, M. J., and Goedert, M. (1998). Tau proteins with FTDP-17 mutations have a reduced ability to promote microtubule assembly. FEBS Lett 437, 207-210.
Jacobsen, J. S., Wu, C. C., Redwine, J. M., Comery, T. A., Arias, R., Bowlby, M., Martone, R., Morrison, J. H., Pangalos, M. N., Reinhart, P. H., and Bloom, F. E. (2006). Early-onset behavioral and synaptic deficits in a mouse model of Alzheimer's disease. Proc Natl Acad Sci USA 103, 5161-5166.
Kins, S., and Beyreuther, K. (2006). Teasing out the tangles. Nat Med 12, 764-765; discussion 765.
Lesne, S., Koh, M. T., Kotilinek, L., Kayed, R., Glabe, C. G., Yang, A., Gallagher, M., and Ashe, K. H. (2006). A specific amyloid-beta protein assembly in the brain impairs memory. Nature 440, 352-357.
Li, X., An, W. L., Alafuzoff, I., Soininen, H., Winblad, B., and Pei, J. J. (2004). Phosphorylated eukaryotic translation factor 4E is elevated in Alzheimer brain. Neuroreport 15, 2237-2240.
Mandelkow, E. M., Biernat, J., Drewes, G., Gustke, N., Trinczek, B., and Mandelkow, E. (1995). Tau domains, phosphorylation, and interactions with microtubules. Neurobiol Aging 16, 355-362; discussion 362-353.
Mount, C., and Downton, C. (2006). Alzheimer disease: progress or profit? Nat Med 12, 780-784.
Roberson, E., and Mucke, L. (2006). 100 years and counting: prospects for defeating alzheimer's disease. Science 314, 781-784.
Santacruz, K., Lewis, J., Spires, T., Paulson, J., Kotilinek, L., Ingelsson, M., Guimaraes, A., DeTure, M., Ramsden, M., McGowan, E., et al. (2005). Tau suppression in a neurodegenerative mouse model improves memory function. Science 309, 476-481.
Ueda, T., Watanabe-Fukunaga, R., Fukuyama, H., Nagata, S., and Fukunaga, R. (2004). Mnk2 and Mnk1 are essential for constitutive and inducible phosphorylation of eukaryotic initiation factor 4E but not for cell growth or development. Mol Cell Biol 24, 6539-6549.
Lee, V. M., Goerder M., and Trojanowski, J. Q. (2001), Neurodegenerative tauopathies. Annu. Rev. Neurosci. 24. 1121-1159.

1. Use of a modulator of Mnk1 and/or Mnk2 kinase for the manufacture of an agent for the diagnosis, alleviation, treatment and/or prevention of a tauopathy.

2. Use of claim 1, wherein the modulator is an antibody or antibody fragment against Mnk1 and/or Mnk2 kinase, an antisense molecule, a ribozyme or an RNAi molecule and/or a low molecular weight organic molecule.

3. Use of claim 1, wherein the modulator is an inhibitor.

4. The use of claim 1, wherein the modulator is a thienopyrimidine, a pyrazolopyrimidine or a pyrrolopyrimidine compound or a pharmaceutically acceptable salt thereof.

5. The use of claim 1, wherein the inhibitor is the compound EDJ101401 or a pharmaceutically acceptable salt thereof.

6. The use of claim 1, wherein the inhibitor is the compound EDJ100869 or a pharmaceutically acceptable salt thereof.

7. The use of claim 1, wherein the tauopathy is selected from disorders showing a coexistence of tau-containing intracellular neurofibrillary tangles (NFTs) and amyloid-β-containing plaques.

8. The use of claim 1, wherein the tauopathy is selected from the group consisting of Alzheimer's disease, Creutzfeldt-Jakob disease, dementia pugilistica, Down's syndrome, Gerstmann-Sträussler-Sheinker disease, inclusion-body myositis, prion protein cerebral amyloid angiopathy.

9. The use of claim 1, wherein the tauopathy is selected from disorders without distinct amyloid-β containing plaques.

10. The use of claim 1, wherein the tauopathies are selected from the group consisting of frontotemporal dementia (FTD), frontotemporal dementia with parkinsonism linked to chromosome 17 (FTDT-17), Pick's disease, tangle-predominant Alzheimer's disease, corticobasal degeneration, amyotrophic lateral sclerosis/parkinsonism-dementia complex, argyrophilic grain dementia, diffuse neurofibrillary tangles with calcification, Hallevorden-Spatz disease, multiple system atrophy, Niemann-Pick disease Type C, progressive subcortial gliosis, progressive supranuclear palsy and subacute sclerosing panencephalitis.

11. The use of claim 1, wherein the tauopathy is Alzheimer's disease.

12. The use of claim 1, wherein the agent is a diagnostic agent.

13. The use of claim 1, wherein the agent is a therapeutic agent.

14. The use of claim 1 for the manufacture of an agent for a monotherapy.

15. The use of claim 1 for the manufacture of an agent for a combination therapy.

16. The use of claim 15 together with at least one further pharmaceutical agent suitable for the alleviation, treatment and/or prevention of a tauopathy.

17. The use of claim 15 together with at least one further pharmaceutical agent suitable for the alleviation, treatment and/or prevention of Alzheimer's disease.

18. The use of claim 17, wherein the further agent is a NMDA antagonist or a acetylcholinesterase inhibitor.

19. The use of claim 18, wherein the NMDA antagonist is memantine and the acetylcholinesterase inhibitor is selected from donepezil, rivastigmin and galantamin.

20. A method of screening of an agent for the diagnosis, alleviation, treatment and/or prevention of a tauopathy comprising the steps
(a) contacting a compound with an at least partially isolated and/or purified Mnk1 and/or Mnk2 kinase,
(b) determining the activity of Mnk1 and/or Mnk2 kinase on phosphorylation of tau protein, and
(c) selecting a compound which reduces the activity of Mnk1 and/or Mnk2 kinase.

21. The method of screening of claim 17, wherein the tau protein is a human tau protein.

22. The method of screening of claim 17, wherein the activity of the Mnk1 and/or Mnk2 kinase in steps (b) and (c) is determined by measuring the phosphorylation of the tau protein on the residues Ser262 and/or Ser356.

23. A method of screening for an agent for the diagnosis, alleviation, treatment and/or prevention of a tauopathy comprising the steps of
(a) providing a cell capable of expressing Mnk1 and/or Mnk2 kinase or/and providing a brain extract containing Mnk1 and/or Mnk2 kinase,
(b) contacting a compound with the cell and/or the brain extract,
(c) determining the amount and/or the activity of Mnk1 and/or Mnk2 kinase and
(d) selecting a compound which reduces the amount or/and the activity of Mnk1 and/or Mnk2 kinase.

24. The method of screening of claim 20, wherein the activity of Mnk1 and/or Mnk2 kinase in steps (c) and (d) is determined by the degree of phosphorylation of the tau protein.

25. The method of screening of claim 24, wherein the tau is a human tau protein.

26. The method of screening of claim 23, wherein the activity of the Mnk1 and/or Mnk2 kinase in steps (c) and (d) is determined by measuring the phosphorylation of the tau protein on the residues Ser262 and/or Ser356.

27. Method for the diagnosis, alleviation, treatment and/or prevention of a tauopathy comprising administering to a subject in need thereof a pharmaceutically effective amount of a modulator of Mnk1 and/or Mnk2 kinase.
